(19) United States
(12) Patent Application Publication
(10) Pub. No.: US 2025/0255932 A1
(43) Pub. Date: Aug. 14, 2025

(54) Granulocyte-Colony Stimulating Factor For The Promotion Of Scarless Tissue Regeneration

(71) Applicants: The United States Government As Represented By The Department of Veterans Affairs, Washington, DC (US); The Trustees Of The University Of Pennsylvania, Philadelphia, PA (US)

(72) Inventor: Thomas H. Leung, Philadelphia, PA (US)

(21) Appl. No.: 19/049,759

(22) Filed: Feb. 10, 2025

Related U.S. Application Data
(60) Provisional application No. 63/551,206, filed on Feb. 8, 2024.

(51) Int. Cl. A61K 38/19; A61K 31/192; A61K 31/437; A61K 36/53; A61K 45/06; A61P 17/02

(52) U.S. Cl. CPC A61K 38/193; A61K 31/192; A61K 31/437; A61K 36/53; A61K 45/06; A61P 17/02

(57) ABSTRACT

Mammals typically heal with fibrotic scars. Treatments to regenerate human skin and hair without a scar remain elusive. Mice lacking C-X-C motif chemokine receptor 2 (CXCR2-KO) displayed robust and complete tissue regeneration across three different injury models, including skin, hair follicle, and cartilage. Remarkably, wild type mice receiving plasma from CXCR2-KO mice through parabiosis or injections healed wounds scarlessly. A comparison of circulating proteins using multiplex ELISA revealed a 24-fold higher plasma level of granulocyte-colony stimulation factor (G-CSF) in CXCR2-KO blood. Local injections of G-CSF into WT mouse wound beds reduced scar formation and increased hair follicle regeneration by 6-fold. G-CSF directly polarized macrophages into an anti-inflammatory phenotype, and both CXCR2-KO and G-CSF-treated mice recruited more anti-inflammatory macrophages into injured areas. These results improve our molecular understanding of scarless tissue regeneration and introduce a new therapeutic approach for cutaneous wounds and hair regeneration. Provided are compositions and methods relating to treating wound healing pathologies.

### CROSS-REFERENCE TO RELATED APPLICATIONS

This application claims the benefit of U.S. Provisional Patent Application No. 63/551,206, filed Feb. 8, 2024, which is incorporated by reference herein in its entirety.

### STATEMENT REGARDING FEDERALLY SPONSORED RESEARCH

This invention was made with government support under 101RX002701 awarded by the Department of Veterans Affairs and R01AR079483 awarded by the National Institutes of Health. The government has certain rights in the invention.

### BACKGROUND

Mice and humans generally heal skin wounds with a fibrotic scar within several weeks (1-5). Scar formation obliterates the native issue architecture and repaired skin lacks accessory organs, including hair and sebaceous glands. Under specific circumstances, mice and humans may heal cutaneous injuries with scarless tissue regeneration, which heals tissues to their original architecture, with return of accessory skin organs (3-6). These findings suggest that the molecular mechanisms driving tissue regeneration remain conserved. Regenerative medicine is charged to find methods to promote scarless tissue regeneration.

Mice lacking chemokine receptor CXCR2 (henceforth known as CXCR2-KO) exhibit decreased immunity against bacterial infections and defective neutrophil chemotaxis, which results in paradoxically increased circulating neutrophils due to defective retention of neutrophils within the bone marrow (7-9). One study showed that non-stented back wounds on CXCR2-KO mice closed with slower speed compared to control mice but did not assess scar formation (10). More recently, non-stented mouse skin wounds were shown to heal primarily by “wound contraction” due to an additional muscle layer rather than “wound healing” (11). Thus, the role of CXCR2 in wound healing remains largely unexplored.

What is needed is need are molecules, compositions and pharmaceutical compositions and methods for treating wound healing pathologies and/or promoting wound healing in a subject in need thereof. Also needed are methods of regenerating skin tissue and reducing scar formation in a subject in need thereof.

### BRIEF SUMMARY

Disclosed herein are pharmaceutical compositions comprising Granulocyte Colony-Stimulating Factor (G-CSF), a G-CSF receptor agonist or a combination thereof and a pharmaceutically acceptable carrier, excipient or diluent.

Disclosed herein are methods for treating wound healing pathologies or for promoting wound healing in a subject having a wound healing pathology, the method comprising the step of administering to the subject a composition comprising a therapeutically effective amount of Granulocyte Colony-Stimulating Factor (G-CSF), a G-CSF receptor agonist or a combination thereof.

Disclosed herein are methods of regenerating tissue in a subject in need thereof, the method comprising the step of administering to the subject a composition comprising a therapeutically effective amount of Granulocyte Colony-Stimulating Factor (G-CSF), a G-CSF receptor agonist or a combination thereof.

Disclosed herein are methods of reducing scar formation to a subject in need thereof, the method comprising the step of administering to the subject a composition comprising a therapeutically effective amount of Granulocyte Colony-Stimulating Factor (G-CSF), a G-CSF receptor agonist or a combination thereof.

Disclosed herein are methods of promoting or increasing hair follicle regeneration in a subject in need thereof, the method comprising the step of administering to the subject a composition comprising a therapeutically effective amount of Granulocyte Colony-Stimulating Factor (G-CSF), a G-CSF receptor agonist or a combination thereof.

Disclosed herein are methods of enhancing neutrophil accumulation to a target site in a subject in need thereof, the method comprising the step of administering to the subject a composition comprising a therapeutically effective amount of Granulocyte Colony-Stimulating Factor (G-CSF), a G-CSF receptor agonist or a combination thereof.

Disclosed herein are methods of polarizing infiltrating macrophages into an anti-inflammatory phenotype in a subject in need thereof, the method comprising the step of administering to the subject a composition comprising a therapeutically effective amount of Granulocyte Colony-Stimulating Factor (G-CSF), a G-CSF receptor agonist or a combination thereof.

Additional advantages of the disclosed method and compositions will be set forth, in part, in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIGS. 1A-1J. show that CXCR2-deficient mice promote complete tissue regeneration (A) Representative photographs and percentage of wound closure in wildtype (WT, n=5), heterozygous (HET, n=12), and CXCR2-KO-homozygous (KO, dashed red line, n=6) mouse cars. Dotted circle represents original 2 mm hole. 2-way ANOVA with KO compared to WT or heterozygous. (B) Representative hematoxylin & eosin stained tissue from WT and CXCR2-KO mouse cars. Original wound size denoted by black bar. Higher magnification images from boxed areas. Black arrows indicated regenerated skin appendages. (C) Representative photographs and percentage of fibrosis assessed by picrosirius red staining in WT (n=16) and KO (n=14) wounds. Unpaired Student's two-tailed t-test. (D) Representative photographs and percentage of wound closure in WT (n=8) and CXCR2-KO (n=16) stented back wounds. 2-way ANOVA. (E) Representative Trichrome stained tissue from WT and CXCR2-KO back wounds. Scar length denoted by black line. Higher magnification images from boxed areas. (F) Scar size measured from histology sections in WT (n=6) and CXCR2-KO wounds (n=5). Unpaired Student's two-tailed t-test. (G) Wound fibrosis assessed by picrosirius red staining in WT (n=4) and CXCR2-KO wounds (n=5). Unpaired Student's two-tailed t-test. (H) Representative photographs and quantification of hair follicle regeneration in WIHN from WT (n=6) and CXCR2-KO wounds (n=12). Unpaired Student's two-tailed t-test. (I) Ear hole closure in littermate WT control (n=8) and IL-17−/−; CXCR2−/−double-knockout mice (n=5). 2-way ANOVA. (J) Ear hole closure in littermate WT control (n=14) and CXCR2−/−(n=11) mice treated with antibiotics. 2-way ANOVA. Scale bars, 100 μM. * P<0.05, **P<0.01, ***P<0.001, and ****P<0.0001. Mean±SEM are plotted.

FIGS. 2A-21 show cell-type specific CXCR2-KO mice exhibit partial tissue regeneration (A) UMAP depicting WT (n=2) and CXCR2-KO (n=3) wounded skin. (B) Dot plot demonstrating levels and percent of cells expressing Cxcr2. (C) Representative image and quantification of immunofluorescence for neutrophils (Ly6G+) in WT and CXCR2-KO wound-edge skin at day 3 (n=5 and 3 for WT and KO respectively) and day 7 (n=2 and 4 for WT and KO respectively). Unpaired two-tailed Student's 1-test. (D) Ear hole closure in control and cell-specific CXCR2-KO mice: keratinocyte (K14-Cre; CXCR2^{f/f}, n=16 and 20 for control and KO respectively), fibroblasts (Coll-Cre-ER; CXCR2^{f/f}, n=8), myeloid cells (neutrophils and macrophages, LysM-Cre; CXCR2^{f/f}, n=7). 2-way ANOVA. (E) Pseudotime trajectory analysis of neutrophils from WT and CXCR2-KO mice. Each dot represents a cell. The left panel demonstrates kinetics, and the right panel illustrates sample origin. (F) Differentially expressed genes identified in pseudotime analysis. (G) Representative images of immunofluorescence detecting neutrophils (Ly6G) and neutrophil extracellular traps (citrullinated-H3, H3-Cit; myeloperoxidase, MPO; neutrophil elastase, NE) in WT and CXCR2-KO mice. n=5. (H) Representative image of immunofluorescence detecting neutrophils (Ly6G) in PADI4-KO mice. n=3. (I) Ear hole closure in control and PADI4-KO mice. n=10. 2-way ANOVA. Scale bars, 100 μM.

FIGS. 3A-3G show increased anti-inflammatory macrophages in injured CXCR2-KO skin (A) UMAP depicting sub-clustering of immune cells. (B) Global ligand-receptor scatter plots measuring incoming and outgoing signals. (C) Dot plot of differential gene expression between WT and CXCR2-KO macrophages. (D) Gene Set Enrichment analysis of macrophage populations in WT and CXCR2-KO wounded skin. (E) Analysis of cell-to-cell interactions between neutrophils (N), monocytes (Mon), and macrophages (Mac) in WT (1^{st} and 3^{rd} columns) and CXCR2-KO (2^{nd} and 4^{th} columns) wounded skin at day 3 post-injury. (F) Representative images and (G) quantification of immunofluorescence of WT and CXCR2-KO wounded skin for CD80 (n=4), COX2 (n=4), CD163 (n=12 for WT and n=11 for KO), MRC1 (n=10 for WT and n=6 for KO), and ARG1 (n=10 for WT and n=6 for KO). Unpaired two-tailed Student's t-test. Scale bars, 100 μM. * P<0.05, **P<0.01, ***P<0.01. Mean±SEM are plotted.

FIGS. 4A-4J show G-CSF is sufficient to reduce scarring and to promote hair follicle regeneration (A) Parabiosis between WT: WT (n=4, black solid line), CXCR2KO: CXCR2KO (n=3, red dotted line), and WT: CXCR2-KO mice (n=4, blue dotted line). Percentage of ear hole closure. 2-way ANOVA comparing WT: KO pairs to WT: WT pairs. (B) WT (n=6) or CXCR2-KO (n=7) plasma was injected daily into the wound bed of WT mice undergoing WIHN for the first 3 days after injury. Representative photographs and quantification of hair follicles. Unpaired two-tailed Student's t-test. (C) ELISA measuring cytokine expression in injured WT (n=4 for day 3; n=3 for day 7) and CXCR2-KO (n=6 for day 3; n=3 for day 7) plasma. Unpaired two-tailed Student's 1-test. (D) G-CSF (n=9) or PBS (control, n=7) was injected daily into the wound bed of WT mice undergoing WIHN for first 3 days after injury. Representative photographs and quantification of hair follicles. Unpaired two-tailed Student's t-test. (E) Representative photographs and quantification of scar size of G-CSF (n=8) or PBS (control, n=3)-treated stented back wounds at day 28 after injury. (F) Representative trichrome stained tissue sections from G-CSF or PBS-treated stented back wounds. Black line highlights scar size. (G) Quantification of scar diameter for G-CSF (n=6) or PBS-treated (n=3) mice. (H) Wound fibrosis assessed by picrosirius red staining in G-CSF (n=14 sections) or PBS-treated (n=10 sections). Unpaired Student's two-tailed t-test. (I) Gene expression levels (FPKM) for monocytes treated with and without G-CSF (n=2 for PBS; n=3 for G-CSF). (J) Representative photographs and quantification of CD163 in G-CSF-(n=5 sections) or PBS-treated (n=7 sections) mice and MRC1 in PBS or G-CSF-treated (n=15 sections) mice. Unpaired Student's two-tailed t-test. Scale bars, 100 μM. * P<0.05, **P<0.01, ***P<0.001. Mean±SEM are plotted.

FIGS. 5A-5C show CXCR2-deficient mice exhibit complete tissue regeneration (A) Representative immunofluorescence of tissue sections from wounded WT and CXCR2-KO mouse cars for cell death (TUNEL) cell proliferation (Ki67) and angiogenesis (CD31). Scale bars, 100 μM. (B) Additional Trichrome stained tissue sections from WT and CXCR2-KO back wounds. Injured area indicated by black line. Higher magnification images from boxed areas. Scale bars, 100 μM. (C) Representative immunofluorescence of wound skin from the WIHN depicting neogenic hair follicles (Krt14+, Krt6+). Scale bars, 100 μM.

FIGS. 6A-6H show cell-type specific CXCR2-KO mice exhibit partial tissue regeneration (A) UMAP plot of single-cell RNA-sequencing data of wounded skin of wild-type (WT) and CXCR2-KO mice at different time points. (B) Bar plot showing relative contribution of different cell types in each single-cell RNA-sequencing skin sample. VE, Vascular endothelium; LE, lymphatic endothelium; DC, Dendritic Cell; Bas.Epi, Basal Epithelial; SuBas.Epi, Suprabasal Basal Epithelial; Prol.Epi, Proliferative Epithelial; HF, Hair follicle; Sm.Mus, Smooth muscle cell; Seb.Glnd, Sebaceous gland. (C) Dot plot of key marker genes for each cell type. Color scale represents gene expression, and dot size represents percentage of cells expressing the marker gene. (D) UMAP plots depicting normalized expression of Cxcr2 transcript in WT and CXCR2-KO wounded skin at different time points. Circle highlights neutrophils. (E) Flow cytometry gating strategy for mouse neutrophils. (F) Quantification of neutrophils in WT and CXCR2-KO wounded skin by flow cytometry. n=2 for day 0 and day 7 groups; n=3 for day 3 groups. Unpaired two-tailed Student's t-test. ***P<0.001. (G) CXCR2 transcript levels in control and cell-type specific CXCR2-KO mice. n=3. **P<0.01. Unpaired two-tailed Student's t-test. (H) Pseudotime trajectory analysis of neutrophils from WT and CXCR2-KO mice. Each dot represents a cell.

FIGS. 7A-7C show increased anti-inflammatory macrophages in injured CXCR2-KO mouse skin (A) UMAP plot of subclustered immune cells of wounded skin of wild-type (WT) and CXCR2-KO mice at different time points. (B) Dot plot of key marker genes for each cell type. The color scale represents gene expression, and the dot size represents the percentage of cells expressing the marker gene. (C) Dot plot demonstrating average expression and percent of cells expressing specific cytokines in WT and CXCR2-KO.

FIGS. 8A-8E show G-CSF is sufficient to reduce scarring and to promote hair follicle regeneration (A) ELISA comparing cytokine expression in plasma from injured WT (n=4 for day 3; n=3 for day 7) and CXCR2-KO (n=6 for day 3; n=3 for day 7) mice. (B) ELISA comparing cytokine expression in wound-edge tissue from injured WT (n=4 for day 3; n=3 for day 7) and CXCR2-KO (n=6 for day 3; n=3 for day 7) mice. (C) Additional histological photographs of scar size of G-CSF or PBS-treated stented back wounds on WT mice at day 28 after injury. Scar size indicated on the panel. (D) Dot plot demonstrating average expression and percent of cells expressing specific cytokines in WT and CXCR2-KO. (E) Representative images of WT and CXCR2-KO wounded skin for F4/80 and G-CSFR (n=4). Scale bars, 100 μM.

FIGS. 9A-9B show TRPA1 activation synergizes with CXCR2-KO mice to drive tissue regeneration. (A) CXCR2-KO mice treated with imiquimod (IMQ) or Cetaphil control (Ceta). *p<0.05; **p<0.01. (B) % mice achieving complete ear-hole closure.

### DETAILED DESCRIPTION

The disclosed method and compositions may be understood more readily by reference to the following detailed description of particular embodiments and the Example included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

### A. Definitions

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a G-CSF receptor agonist” includes a plurality of such G-CSF receptor agonist molecules, reference to “the composition” is a reference to one or more compositions and equivalents thereof known to those skilled in the art, and so forth.

The word “or” as used herein means any one member of a particular list and also includes any combination of members of that list.

As used herein, the term “agonist” refers to a molecule that activates a receptor to produce a biological response.

As used herein, the term “subject” or “patient” can be used interchangeably and refer to any organism to which a composition of this invention may be administered, e.g., for experimental, diagnostic, and/or therapeutic purposes. Typical subjects include animals (e.g., mammals such as non-human primates, and humans; avians; domestic household or farm animals such as cats, dogs, sheep, goats, cattle, horses and pigs; laboratory animals such as mice, rats and guinea pigs; rabbits; fish; reptiles; zoo and wild animals). Typically, “subjects” are animals, including mammals such as humans and primates; and the like.

As used herein, “treat” is meant to mean administer one of the disclosed compositions to a subject, such as a human or other mammal (for example, an animal model), that has a skin injury or skin disease, in order to prevent or delay a worsening of the effects of the disease or condition, or to partially or fully reverse the effects of the injury or disease. Treatment need not mean that the wound, disease, disorder, or condition is totally cured. A useful composition herein needs only to reduce the severity of a skin wound, disease, disorder, or to condition, reduce the severity of symptoms associated therewith, provide improvement to a patient's quality of life, or delay, prevent, or inhibit the onset of a skin disease, disorder, or condition.

As used herein, “effective amount” is meant to mean a sufficient amount of one or more of the molecules, compositions or pharmaceutical compositions disclosed herein to provide the desired effect. For example, an effective amount of one or more of the molecules, compositions or pharmaceutical compositions disclosed herein can be an amount that provides a therapeutic affect and provides sustained therapeutic effects after withdrawal of the treatment. An effective amount of one or more of the molecules, compositions or pharmaceutical compositions disclosed herein can be an amount that is able to cause a benefit illustrated by, for example, promoting healing of tendon injuries, speeding up recovery time, improving biomechanical properties (such as strength or toughness) of the bone, cartilage and/or tendon, and/or improving tissue organization of bone, cartilage and/or tendon, as well as an amount that allows for a sustained therapeutic effect after withdrawal of the molecule, composition or pharmaceutical composition. The exact amount required will vary from subject to subject, depending on the species, age, and general condition of the subject, the severity of disease (or underlying genetic defect) that is being treated, the particular compound used, its mode of administration, and the like. Thus, it is not possible to specify an exact “effective amount.” However, an appropriate “effective amount” may be determined by one of ordinary skill in the art using only routine experimentation.

As used herein, the terms “administering” and “administration” refer to any method of providing a disclosed composition to a subject. Such methods are well known to those skilled in the art and include, but are not limited to: oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition. In an aspect, the skilled person can determine an efficacious dose, an efficacious schedule, or an efficacious route of administration for a disclosed composition or a disclosed protein so as to treat a subject or induce an immune response. In an aspect, the skilled person can also alter or modify an aspect of an administering step so as to improve efficacy of a disclosed composition.

In various aspects, the present compositions may be used in combination with an additional pharmaceutical dosage form to enhance their effectiveness in treating a skin disease, disorder, or condition. In this regard, the present compositions may be administered as part of a regimen additionally including another pharmaceutical, and/or pharmaceutical dosage form known in the art as effective for the treatment of a skin disorder. Accordingly, the additional active ingredient or additional pharmaceutical dosage form can be applied to a patient either directly or indirectly, and concomitantly or sequentially, with the compositions described herein. In some embodiments, the present compositions may be administered as part of a regimen further comprising administering, for example by intralesional injection, a corticosteroid, such as cortisone, to the subject.

As used herein, the term “abrasion” refers to a partial thickness wound caused by damage to the skin. It can be superficial involving only the epidermis to deep, involving the deep dermis. As used herein, the term “avulsion” is a wound in which all layers of the skin have been torn away, exposing the underlying structures (i.e., subcutaneous tissue, muscle, tendons, or bone). As used herein, the term “burn” refers to damage to the skin or tissues caused by heat, electricity, chemicals, radiation, or sunlight. As used herein, the term “laceration” refers to a cut or tear in the skin that is caused by blunt or sharp trauma. As used herein, the term “surgical wound” refers to an incision or cut made in the skin or underlying tissues during a surgical procedure.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinence of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

As used herein, the term “comprising” can include the aspects “consisting of” and “consisting essentially of.”

### B. Compositions

Disclosed herein are pharmaceutical compositions comprising Granulocyte Colony-Stimulating Factor (G-CSF), a G-CSF receptor agonist or a combination thereof and a pharmaceutically acceptable carrier, excipient or diluent.

In some aspects, the pharmaceutical composition comprises G-CSF and a pharmaceutically acceptable carrier, excipient or diluent. In some aspects, the G-CSF is filgrastim or lenograstim. In some aspects, the G-CSF is the 174 amino acid form of G-CSF. In some aspects, the G-CSF is the 177 amino acid form of G-CSF. In some aspects, the pharmaceutical composition comprises PEGylated G-CSF and a pharmaceutically acceptable carrier, excipient or diluent.

In some aspects, the G-CSF receptor agonist is Myelopoietin (an engineered chimeric IL-3 and G-CSF receptor agonist), LG7455 or GCSF receptor agonist antibodies, such as those described in WO 1999/055735 A1.

In some aspects, the pharmaceutical composition is formulated in a solid, semi-solid or liquid dosage form. In some aspects, the pharmaceutical composition is formulated in a topical ointment, cream, lotion or spray.

In some aspects, the pharmaceutical composition further comprises a CXCR2 inhibitor. In some aspects, the CXCR2 inhibitor is SB 265610, AZD5069, NAVARIXIN (SCH-527123), SB225002, DANIRIXIN (GSK1325756), REPARIXIN (REPERTAXIN), SX-682 or a combination thereof.

In some aspects, the pharmaceutical composition further comprises a transient receptor potential ankyrin 1 (TRPA1) receptor agonist. In some aspects, the TRPA1 receptor agonist is imiquimod, allyl isothiocyanate (AITC), Cinnamaldehyde, acryloins, delta9-tetrahydrocannabinol (THC), methyl syringate, bradykinin, rosemary extract, carnosic acid or a combination thereof. In some aspects, the TRPA1 receptor agonist is imiquimod.

### C. Methods

### 1. Methods for Treating Wound Healing Pathologies or for Promoting Wound Healing

Disclosed herein are methods for treating wound healing pathologies or for promoting wound healing in a subject having a wound healing pathology comprising administering any one of the disclosed molecules, compositions or pharmaceutical compositions to the subject.

Disclosed herein are methods for treating wound healing pathologies or for promoting wound healing in a subject having a wound healing pathology comprising administering to the subject a composition comprising a therapeutically effective amount of Granulocyte Colony-Stimulating Factor (G-CSF), a G-CSF receptor agonist or a combination thereof. In some aspects of the methods for treating wound healing pathologies or for promoting wound healing in a subject in need thereof, the composition comprises G-CSF. In some aspects of the methods for treating wound healing pathologies or for promoting wound healing in a subject in need thereof, the G-CSF is filgrastim or lenograstim. In some aspects of the methods for treating wound healing pathologies or for promoting wound healing in a subject in need thereof, the G-CSF is the 174 amino acid form of G-CSF. In some aspects of the methods for treating wound healing pathologies or for promoting wound healing in a subject in need thereof, the G-CSF is the 177 amino acid form of G-CSF. In some aspects of the methods for treating wound healing pathologies or for promoting wound healing in a subject in need thereof, the G-CSF receptor agonist is myelopoietin, LG7455, a GCSF receptor agonist antibody or a combination thereof.

In some aspects of the methods for treating wound healing pathologies or for promoting wound healing in a subject in need thereof, the step of administering is performed during spreading stage of scar formation.

In some aspects of the methods for treating wound healing pathologies or for promoting wound healing in a subject in need thereof, the Granulocyte Colony-Stimulating Factor (G-CSF), a G-CSF receptor agonist or a combination thereof is administered by topical administration. In some aspects of the methods for treating wound healing pathologies or for promoting wound healing in a subject in need thereof, the composition is a topical ointment, cream, lotion or spray.

In some aspects of the methods for treating wound healing pathologies or for promoting wound healing in a subject in need thereof, the Granulocyte Colony-Stimulating Factor (G-CSF), a G-CSF receptor agonist or a combination thereof is administered by intradermal or sub epidermal administration.

In some aspects of the methods for treating wound healing pathologies or for promoting wound healing in a subject in need thereof, the Granulocyte Colony-Stimulating Factor (G-CSF), a G-CSF receptor agonist or a combination thereof is administered by intralesional injection.

In some aspects, the wound healing pathologies can be ulcers, chronic wounds, keloids, hypertrophic scars, contractures, dehiscence, or pathological inflammation. In some aspects, the wound can be an acute wound, a thermal wound (e.g. a burn), chemical wound, a penetrating wound, an open wound or a closed wound. In some aspects, the wound can be an incision, laceration, abrasion, avulsion, puncture wound, penetration wound, critical wound, hematoma, crush injury, or a fracture. In some aspects of the methods for treating wound healing pathologies or for promoting wound healing in a subject in need thereof, the wound is an abrasion, an avulsion, a burn, a laceration or a surgical wound.

In some aspects of the methods for treating wound healing pathologies or for promoting wound healing in a subject in need thereof, the method further comprises administering a CXCR2 inhibitor. In some aspects of the methods for treating wound healing pathologies or for promoting wound healing in a subject in need thereof, the CXCR2 inhibitor is SB 265610, AZD5069, NAVARIXIN (SCH-527123), SB225002, DANIRIXIN (GSK1325756), REPARIXIN (REPERTAXIN), SX-682 or a combination thereof.

In some aspects of the methods for treating wound healing pathologies or for promoting wound healing in a subject in need thereof, the method further comprises administering a transient receptor potential ankyrin 1 (TRPA1) receptor agonist. In some aspects of the methods for treating wound healing pathologies or for promoting wound healing in a subject in need thereof, the TRPA1 receptor agonist is imiquimod, allyl isothiocyanate (AITC), Cinnamaldehyde, acryloins, delta9-tetrahydrocannabinol (THC), methyl syringate, bradykinin, rosemary extract, carnosic acid or a combination thereof. In some aspects of the methods for treating wound healing pathologies or for promoting wound healing in a subject in need thereof, the TRPA1 receptor agonist is imiquimod.

In some aspects of the methods for treating wound healing pathologies or for promoting wound healing in a subject in need thereof, the composition further comprises rosemary extract or carnosic acid. Disclosed herein are methods for treating wound healing pathologies or for promoting wound healing in a subject having a wound healing pathology comprising administering to the subject a composition comprising a therapeutically effective amount of Granulocyte Colony-Stimulating Factor (G-CSF), a G-CSF receptor agonist or a combination thereof, wherein the method further comprises administering rosemary extract or carnosic acid to the subject.

### 2. Methods of Regenerating Tissue

Disclosed herein are methods of regenerating tissue in a subject in need thereof comprising administering any one of the disclosed molecules, compositions or pharmaceutical compositions to the subject.

Disclosed herein are methods of regenerating tissue in a subject in need thereof comprising administering to the subject a composition comprising a therapeutically effective amount of Granulocyte Colony-Stimulating Factor (G-CSF), a G-CSF receptor agonist or a combination thereof. In some aspects of the methods of regenerating tissue in a subject in need thereof, the composition comprises G-CSF. In some aspects methods of regenerating tissue in a subject in need thereof, the G-CSF is filgrastim or lenograstim. In some aspects methods of regenerating tissue in a subject in need thereof, the G-CSF is the 174 amino acid form of G-CSF. In some aspects methods of regenerating tissue in a subject in need thereof, the G-CSF is the 177 amino acid form of G-CSF. In some aspects of the methods of regenerating tissue in a subject in need thereof, the G-CSF receptor agonist is myelopoietin, LG7455, a GCSF receptor agonist antibody or a combination thereof.

In some aspects of the methods of regenerating tissue in a subject in need thereof, the Granulocyte Colony-Stimulating Factor (G-CSF), a G-CSF receptor agonist or a combination thereof is administered by topical administration. In some aspects of the methods of regenerating tissue in a subject in need thereof, the composition is a topical ointment, cream, lotion or spray.

In some aspects of the methods of regenerating tissue in a subject in need thereof, the Granulocyte Colony-Stimulating Factor (G-CSF), a G-CSF receptor agonist or a combination thereof is administered by intradermal or subepidermal administration.

In some aspects of the methods of regenerating tissue in a subject in need thereof, the Granulocyte Colony-Stimulating Factor (G-CSF), a G-CSF receptor agonist or a combination thereof is administered by intralesional injection.

In some aspects of the methods of regenerating tissue in a subject in need thereof, the method further comprises administering a CXCR2 inhibitor. In some aspects of the methods of regenerating tissue in a subject in need thereof, the CXCR2 inhibitor is SB 265610, AZD5069, NAVARIXIN (SCH-527123), SB225002, DANIRIXIN (GSK1325756), REPARIXIN (REPERTAXIN), SX-682 or a combination thereof.

In some aspects of the methods of regenerating tissue in a subject in need thereof, the method further comprises administering a transient receptor potential ankyrin 1 (TRPA1) receptor agonist. In some aspects of the methods of regenerating tissue in a subject in need thereof, the TRPA1 receptor agonist is imiquimod, allyl isothiocyanate (AITC), Cinnamaldehyde, acryloins, delta9-tetrahydrocannabinol (THC), methyl syringate, bradykinin, rosemary extract, carnosic acid or a combination thereof. In some aspects of the methods of regenerating tissue in a subject in need thereof, the TRPA1 receptor agonist is imiquimod.

In some aspects of the methods of regenerating tissue in a subject in need thereof, the composition further comprises rosemary extract or carnosic acid. Disclosed herein are methods of regenerating tissue in a subject in need thereof comprising administering to the subject a composition comprising a therapeutically effective amount of Granulocyte Colony-Stimulating Factor (G-CSF), a G-CSF receptor agonist or a combination thereof, wherein the method further comprises administering rosemary extract or carnosic acid to the subject.

### 3. Method of Reducing Scar Formation

Disclosed herein are methods of reducing scar formation in a subject in need thereof comprising administering any one of the disclosed molecules, compositions or pharmaceutical compositions to the subject.

Disclosed herein are methods of reducing scar formation in a subject in need thereof comprising administering to the subject a composition comprising a therapeutically effective amount of Granulocyte Colony-Stimulating Factor (G-CSF), a G-CSF receptor agonist or a combination thereof. In some aspects of the methods of reducing scar formation in a subject in need thereof, the composition comprises G-CSF. In some aspects methods of reducing scar formation in a subject in need thereof, the G-CSF is filgrastim or lenograstim. In some aspects methods of reducing scar formation in a subject in need thereof, the G-CSF is the 174 amino acid form of G-CSF. In some aspects methods of reducing scar formation in a subject in need thereof, the G-CSF is the 177 amino acid form of G-CSF. In some aspects of the methods of reducing scar formation in a subject in need thereof, the G-CSF receptor agonist is myelopoietin, LG7455, a GCSF receptor agonist antibody or a combination thereof.

In some aspects of the methods of reducing scar formation in a subject in need thereof, said step of administering is performed during the spreading stage of scar formation.

In some aspects of the methods of reducing scar formation in a subject in need thereof, the Granulocyte Colony-Stimulating Factor (G-CSF), a G-CSF receptor agonist or a combination thereof is administered by topical administration. In some aspects of the methods of reducing scar formation in a subject in need thereof, the composition is a topical ointment, cream, lotion or spray.

In some aspects of the methods of reducing scar formation in a subject in need thereof, the Granulocyte Colony-Stimulating Factor (G-CSF), a G-CSF receptor agonist or a combination thereof is administered by intradermal or subepidermal administration.

In some aspects of the methods of reducing scar formation in a subject in need thereof, the Granulocyte Colony-Stimulating Factor (G-CSF), a G-CSF receptor agonist or a combination thereof is administered by intralesional injection.

In some aspects of the methods of reducing scar formation in a subject in need thereof, the scar formation is from an abrasion, an avulsion, a burn, a laceration or a surgical wound.

In some aspects of the methods of reducing scar formation in a subject in need thereof, the method further comprises administering a CXCR2 inhibitor. In some aspects of the methods of reducing scar formation in a subject in need thereof, the CXCR2 inhibitor is SB 265610, AZD5069, NAVARIXIN (SCH-527123), SB225002, DANIRIXIN (GSK1325756), REPARIXIN (REPERTAXIN), SX-682 or a combination thereof.

In some aspects of the methods of reducing scar formation in a subject in need thereof, the method further comprises administering a transient receptor potential ankyrin 1 (TRPA1) receptor agonist. In some aspects of the methods of reducing scar formation in a subject in need thereof, the TRPA1 receptor agonist is imiquimod, allyl isothiocyanate (AITC), Cinnamaldehyde, acryloins, delta9-tetrahydrocannabinol (THC), methyl syringate, bradykinin, rosemary extract, carnosic acid or a combination thereof. In some aspects of the methods of reducing scar formation in a subject in need thereof, the TRPA1 receptor agonist is imiquimod.

In some aspects of the methods of reducing scar formation in a subject in need thereof, the composition further comprises rosemary extract or carnosic acid. Disclosed herein are methods of reducing scar formation in a subject in need thereof comprising administering to the subject a composition comprising a therapeutically effective amount of Granulocyte Colony-Stimulating Factor (G-CSF), a G-CSF receptor agonist or a combination thereof, wherein the method further comprises administering rosemary extract or carnosic acid to the subject.

### 4. Methods of Promoting or Increasing Hair Follicle Regeneration

Disclosed herein are methods for promoting or increasing hair follicle regeneration in a subject in need thereof comprising administering any one of the disclosed molecules, compositions or pharmaceutical compositions to the subject.

Disclosed herein are methods for promoting or increasing hair follicle regeneration in a subject in need thereof comprising administering to the subject a composition comprising a therapeutically effective amount of Granulocyte Colony-Stimulating Factor (G-CSF), a G-CSF receptor agonist or a combination thereof. In some aspects of the methods for promoting or increasing hair follicle regeneration in a subject in need thereof, the composition comprises G-CSF. In some aspects methods for promoting or increasing hair follicle regeneration in a subject in need thereof, the G-CSF is filgrastim or lenograstim. In some aspects methods for promoting or increasing hair follicle regeneration in a subject in need thereof, the G-CSF is the 174 amino acid form of G-CSF. In some aspects methods for promoting or increasing hair follicle regeneration in a subject in need thereof, the G-CSF is the 177 amino acid form of G-CSF. In some aspects of the methods for promoting or increasing hair follicle regeneration in a subject in need thereof, the G-CSF receptor agonist is myelopoietin, LG7455, a GCSF receptor agonist antibody or a combination thereof.

In some aspects of the methods for promoting or increasing hair follicle regeneration in a subject in need thereof, the Granulocyte Colony-Stimulating Factor (G-CSF), a G-CSF receptor agonist or a combination thereof is administered by topical administration. In some aspects of the methods for promoting or increasing hair follicle regeneration in a subject in need thereof, the composition is a topical ointment, cream, lotion or spray.

In some aspects of the methods for promoting or increasing hair follicle regeneration in a subject in need thereof, the Granulocyte Colony-Stimulating Factor (G-CSF), a G-CSF receptor agonist or a combination thereof is administered by intradermal or subepidermal administration.

In some aspects of the methods for promoting or increasing hair follicle regeneration in a subject in need thereof, the Granulocyte Colony-Stimulating Factor (G-CSF), a G-CSF receptor agonist or a combination thereof is administered by intralesional injection.

In some aspects of the methods for promoting or increasing hair follicle regeneration in a subject in need thereof, the method further comprises administering a CXCR2 inhibitor. In some aspects of the methods for promoting or increasing hair follicle regeneration in a subject in need thereof, the CXCR2 inhibitor is SB 265610, AZD5069, NAVARIXIN (SCH-527123), SB225002, DANIRIXIN (GSK1325756), REPARIXIN (REPERTAXIN), SX-682 or a combination thereof.

In some aspects of the methods for promoting or increasing hair follicle regeneration in a subject in need thereof, the method further comprises administering a transient receptor potential ankyrin 1 (TRPA1) receptor agonist. In some aspects of the methods for promoting or increasing hair follicle regeneration in a subject in need thereof, the TRPA1 receptor agonist is imiquimod, allyl isothiocyanate (AITC), Cinnamaldehyde, acryloins, delta9-tetrahydrocannabinol (THC), methyl syringate, bradykinin, rosemary extract, carnosic acid or a combination thereof. In some aspects of the methods for promoting or increasing hair follicle regeneration in a subject in need thereof, the TRPA1 receptor agonist is imiquimod.

In some aspects of the methods for promoting or increasing hair follicle regeneration in a subject in need thereof, the composition further comprises rosemary extract or carnosic acid. Disclosed herein are methods for promoting or increasing hair follicle regeneration in a subject in need thereof comprising administering to the subject a composition comprising a therapeutically effective amount of Granulocyte Colony-Stimulating Factor (G-CSF), a G-CSF receptor agonist or a combination thereof, wherein the method further comprises administering rosemary extract or carnosic acid to the subject.

### 5. Methods of Enhancing Neutrophil Accumulation to a Target Site

Disclosed herein are methods of enhancing neutrophil accumulation to a target site in a subject in need thereof comprising administering any one of the disclosed molecules, compositions or pharmaceutical compositions to the subject.

In some aspects of the methods of enhancing neutrophil accumulation to a target site in a subject in need thereof, the target site is a wound, such as an abrasion, an avulsion, a burn, a laceration or a surgical wound. In some aspects of the methods of enhancing neutrophil accumulation to a target site in a subject in need thereof, the target site is a wound, such as an abrasion, an avulsion, a burn, a laceration or a surgical wound. In some aspects of the methods of enhancing neutrophil accumulation to a target site in a subject in need thereof, the target site is an ulcer, chronic wound, keloid, hypertrophic scar, contracture, dehiscence, or pathological inflammation. In some aspects of the methods of enhancing neutrophil accumulation to a target site in a subject in need thereof, the target site can be an acute wound, a thermal wound (e.g. a burn), chemical wound, a penetrating wound, an open wound or a closed wound. In some aspects of the methods of enhancing neutrophil accumulation to a target site in a subject in need thereof, the target site can be an incision, laceration, abrasion, avulsion, puncture wound, penetration wound, critical wound, hematoma, crush injury, or a fracture.

Disclosed herein are methods of enhancing neutrophil accumulation to a target site in a subject in need thereof comprising administering to the subject a composition comprising a therapeutically effective amount of Granulocyte Colony-Stimulating Factor (G-CSF), a G-CSF receptor agonist or a combination thereof. In some aspects of the methods of enhancing neutrophil accumulation to a target site in a subject in need thereof, the composition comprises G-CSF. In some aspects methods of enhancing neutrophil accumulation to a target site in a subject in need thereof, the G-CSF is filgrastim or lenograstim. In some aspects methods of enhancing neutrophil accumulation to a target site in a subject in need thereof, the G-CSF is the 174 amino acid form of G-CSF. In some aspects methods of enhancing neutrophil accumulation to a target site in a subject in need thereof, the G-CSF is the 177 amino acid form of G-CSF. In some aspects of the methods of enhancing neutrophil accumulation to a target site in a subject in need thereof, the G-CSF receptor agonist is myelopoietin, LG7455, a GCSF receptor agonist antibody or a combination thereof.

In some aspects of the methods of enhancing neutrophil accumulation to a target site in a subject in need thereof, the Granulocyte Colony-Stimulating Factor (G-CSF), the G-CSF receptor agonist or a combination thereof is administered by topical administration. In some aspects of the methods of enhancing neutrophil accumulation to a target site in a subject in need thereof, the composition is a topical ointment, cream, lotion or spray.

In some aspects of the methods of enhancing neutrophil accumulation to a target site in a subject in need thereof, the Granulocyte Colony-Stimulating Factor (G-CSF), a G-CSF receptor agonist or a combination thereof is administered by intradermal or subepidermal administration.

In some aspects of the methods of enhancing neutrophil accumulation to a target site in a subject in need thereof, the Granulocyte Colony-Stimulating Factor (G-CSF), a G-CSF receptor agonist or a combination thereof is administered by intralesional injection.

In some aspects of the methods of enhancing neutrophil accumulation to a target site in a subject in need thereof, the method further comprises administering a CXCR2 inhibitor. In some aspects of the methods of enhancing neutrophil accumulation to a target site in a subject in need thereof, the CXCR2 inhibitor is SB 265610, AZD5069, NAVARIXIN (SCH-527123), SB225002, DANIRIXIN (GSK1325756), REPARIXIN (REPERTAXIN), SX-682 or a combination thereof.

In some aspects of the methods of enhancing neutrophil accumulation to a target site in a subject in need thereof, the method further comprises administering a transient receptor potential ankyrin 1 (TRPA1) receptor agonist. In some aspects of the methods of enhancing neutrophil accumulation to a target site in a subject in need thereof, the TRPA1 receptor agonist is imiquimod, allyl isothiocyanate (AITC), Cinnamaldehyde, acryloins, delta9-tetrahydrocannabinol (THC), methyl syringate, bradykinin, rosemary extract, carnosic acid or a combination thereof. In some aspects of the methods of enhancing neutrophil accumulation to a target site in a subject in need thereof, the TRPA1 receptor agonist is imiquimod.

In some aspects of the methods of enhancing neutrophil accumulation to a target site in a subject in need thereof, the composition further comprises rosemary extract or carnosic acid. Disclosed herein are methods of enhancing neutrophil accumulation to a target site in a subject in need thereof comprising administering to the subject a composition comprising a therapeutically effective amount of Granulocyte Colony-Stimulating Factor (G-CSF), a G-CSF receptor agonist or a combination thereof, wherein the method further comprises administering rosemary extract or carnosic acid to the subject.

6. Methods of Polarizing Infiltrating Macrophages into an Anti-Inflammatory Phenotype

Disclosed herein are methods of polarizing infiltrating macrophages into an anti-inflammatory phenotype in a subject in need thereof comprising administering any one of the disclosed molecules, compositions or pharmaceutical compositions to the subject.

Disclosed herein are methods of polarizing infiltrating macrophages into an anti-inflammatory phenotype in a subject in need thereof comprising administering to the subject a composition comprising a therapeutically effective amount of Granulocyte Colony-Stimulating Factor (G-CSF), a G-CSF receptor agonist or a combination thereof. In some aspects of the methods of polarizing infiltrating macrophages into an anti-inflammatory phenotype in a subject in need thereof, the composition comprises G-CSF. In some aspects methods of polarizing infiltrating macrophages into an anti-inflammatory phenotype in a subject in need thereof, the G-CSF is filgrastim or lenograstim. In some aspects methods of polarizing infiltrating macrophages into an anti-inflammatory phenotype in a subject in need thereof, the G-CSF is the 174 amino acid form of G-CSF. In some aspects methods of polarizing infiltrating macrophages into an anti-inflammatory phenotype in a subject in need thereof, the G-CSF is the 177 amino acid form of G-CSF. In some aspects of the methods of polarizing infiltrating macrophages into an anti-inflammatory phenotype in a subject in need thereof, the G-CSF receptor agonist is myclopoietin, LG7455, a GCSF receptor agonist antibody or a combination thereof.

In some aspects of the methods of polarizing infiltrating macrophages into an anti-inflammatory phenotype in a subject in need thereof, the Granulocyte Colony-Stimulating Factor (G-CSF), a G-CSF receptor agonist or a combination thereof is administered by topical administration. In some aspects of the methods of polarizing infiltrating macrophages into an anti-inflammatory phenotype in a subject in need thereof, the composition is a topical ointment, cream, lotion or spray.

In some aspects of the methods of polarizing infiltrating macrophages into an anti-inflammatory phenotype in a subject in need thereof, the Granulocyte Colony-Stimulating Factor (G-CSF), a G-CSF receptor agonist or a combination thereof is administered by intradermal or subepidermal administration.

In some aspects of the methods of polarizing infiltrating macrophages into an anti-inflammatory phenotype in a subject in need thereof, the Granulocyte Colony-Stimulating Factor (G-CSF), a G-CSF receptor agonist or a combination thereof is administered by intralesional injection.

In some aspects of the methods of polarizing infiltrating macrophages into an anti-inflammatory phenotype in a subject in need thereof, the method further comprises administering a CXCR2 inhibitor. In some aspects of the methods of polarizing infiltrating macrophages into an anti-inflammatory phenotype in a subject in need thereof, the CXCR2 inhibitor is SB 265610, AZD5069, NAVARIXIN (SCH-527123), SB225002, DANIRIXIN (GSK1325756), REPARIXIN (REPERTAXIN), SX-682 or a combination thereof.

In some aspects of the methods of polarizing infiltrating macrophages into an anti-inflammatory phenotype in a subject in need thereof, the method further comprises administering a transient receptor potential ankyrin 1 (TRPA1) receptor agonist. In some aspects of the methods of polarizing infiltrating macrophages into an anti-inflammatory phenotype in a subject in need thereof, the TRPA1 receptor agonist is imiquimod, allyl isothiocyanate (AITC), Cinnamaldehyde, acryloins, delta9-tetrahydrocannabinol (THC), methyl syringate, bradykinin, rosemary extract, carnosic acid or a combination thereof. In some aspects of the methods of polarizing infiltrating macrophages into an anti-inflammatory phenotype in a subject in need thereof, the TRPA1 receptor agonist is imiquimod.

In some aspects of the methods of polarizing infiltrating macrophages into an anti-inflammatory phenotype in a subject in need thereof, the composition further comprises rosemary extract or carnosic acid. Disclosed herein are methods of polarizing infiltrating macrophages into an anti-inflammatory phenotype in a subject in need thereof comprising administering to the subject a composition comprising a therapeutically effective amount of Granulocyte Colony-Stimulating Factor (G-CSF), a G-CSF receptor agonist or a combination thereof, wherein the method further comprises administering rosemary extract or carnosic acid to the subject.

### 7. Dosing Regimens

Disclosed are dosing regimens comprising at least one treatment cycle of an effective amount of any of the disclosed pharmaceutical compositions.

Disclosed herein are dosing regimens in which the pharmaceutical composition is administered only once.

Disclosed herein are dosing regimens in which the pharmaceutical composition is administered multiple times over a period of time.

Disclosed herein are dosage regiments in which administration of the pharmaceutical composition can occur anywhere from immediately after the wound occurs to months or years after the wound occurs.

Treatment cycles can include the administration of different dosages of molecules, compositions or pharmaceutical compositions as well as administration at different time points. The pharmaceutical compositions can be administered for varying amounts of time for up to 6 months. The pharmaceutical compositions can be administered for varying amounts of time indefinitely. In some instances, the administration can occur for up to one, two, three, four, five or six months. For example, the pharmaceutical composition can be administered once or twice or three times a day for 1, 2, 3, 4, 6, 8, 10, 12, 14, 16, 18, 20, 22, 24, 26 or 52 weeks.

The length of time for each treatment cycle can vary depending on the amount of molecule, composition or pharmaceutical composition administered per dosage. A treatment cycle can include the administration of a molecule, composition or pharmaceutical composition once, twice or three times a day. In some aspects, the molecule, composition or pharmaceutical composition can be administered daily. In some aspects, the molecule, composition or pharmaceutical composition can be administered once every week, once every two weeks or even once a month. In some instances, the molecule, composition or pharmaceutical composition can be administered every two weeks for 4, 6, 8, 10, 12, 14, 16, 18, 20, 22, or 24 weeks. For example, the treatment cycle can include administering a molecule, composition or pharmaceutical composition once a week for four weeks or once every two weeks for up to six months. Thus, each treatment cycle includes an established length of time for administration as well as an established dosing schedule during that time frame.

In one aspect, more than molecule, composition or pharmaceutical composition can be administered during the treatment cycles. The more than one molecule, composition or pharmaceutical composition can be formulated together or in separate compositions. In some instances, one or more molecules, compositions or pharmaceutical compositions is administered in combination with one or more other therapeutic agents, including, but not limited to antibodies, nanobodies, aptamers, liposomes, antioxidants, anti-inflammatory agents, senolytic agents.

### 8. Dose

The dose or dosage of molecule, composition or pharmaceutical composition can vary depending on many factors, such as but not limited to, age, condition, sex and extent of the disease in the patient, route of administration, length of treatment cycle, or whether other drugs are included in the regimen, and can be determined by one of skill in the art.

Effective dosages can be determined empirically, and making such determinations is within the skill in the art. The dosage ranges for the administration of the compositions are those large enough to produce the desired effect in which the disease is treated. For example, the dosage can be an amount effective to provide therapeutic effects and provide or allow for sustained therapeutic effects even after the treatment (e.g. one or more of the pharmaceutical compositions comprising G-CSF or a G-CSF receptor agonist or combination thereof) is withdrawn. The therapeutic effects can be, but are not limited to, an improvement in wound healing or a reduction in scar formation. The therapeutic effects can be measured by wound assessment tools, including imaging techniques, histology, and gene expression changes. These differences may be measured by evaluating for fibrosis (picrosirius red staining, Trichome staining), amount and length of dermal elastic fibers, or collagen composition. Differences may also be measured by the size and length of clinical scars, return of hair follicles and sebaceous glands, and return of pigmentation. See, e.g., Bernatchez S F, Bichel J. The Science of Skin: Measuring Damage and Assessing Risk. Adv Wound Care (New Rochelle). 2023 April; 12 (4): 187-204.

The dosage should not be so large as to cause adverse side effects, such as unwanted cross-reactions, anaphylactic reactions, and the like. The dosage can be adjusted by the individual physician in the event of any counter-indications. Dosage can vary, and can be administered in one or more dose administrations daily, for one or several days. Guidance can be found in the literature for appropriate dosages for given classes of pharmaceutical products.

Suitable dosages include, but are not limited to amounts between 0.01 mg/day and 20 mg/day. For example, disclosed herein are methods involving administering one or more of the disclosed pharmaceutical compositions to a subject, wherein the concentration of active ingredient(s) can be 0.01, 0.1, 0.2, 0.5, 1, 2, 3, 4, 5, 6, 7, 8, 9, 10, 11, 12, 13, 14, 15, 16, 17, 18, 19, or 20%. Doses will depend on the identity of the active ingredient, for example, the identity of the G-CSF or G-CSF receptor agonist. Preparations of G-CSF, for example, may be about 0.1, 0.2, 0.5, 1, 5 or 10%. Preparations of imiquimod, for example, may be at 5%.

The molecule, composition or pharmaceutical composition dose can be administered systemically or locally. The molecule, composition or pharmaceutical composition dose can be administered as a bolus injection or as an infusion over one or more hours.

### 9. Delivery

In the methods described herein, administration or delivery of the molecules, compositions or pharmaceutical compositions can be via a variety of mechanisms. As defined above, disclosed herein are methods of treating, dosing regimens and methods of using those dosing regimens to treat wound healing pathologies or promote wound healing, to regenerate tissue, to reduce scar formation, to promote or increase hair follicle regeneration, to enhance neutrophil accumulation and to polarize infiltrating macrophages into an anti-inflammatory phenotype. The dosing regimens and methods include compositions containing any one or more of the molecules described herein that can also include a carrier such as a pharmaceutically acceptable carrier. For example, disclosed are pharmaceutical compositions, comprising the molecules and compositions disclosed herein, and a pharmaceutically acceptable carrier.

The disclosed pharmaceutical compositions can be in solution or in suspension (for example, incorporated into microparticles, liposomes, or cells).

Any suitable route of administration can be used for the disclosed pharmaceutical compositions. Suitable routes of administration can, for example, include topical, enteral, local, systemic, or parenteral. For example, administration can be epicutaneous, inhalational, enema, conjunctival, eye drops, ear drops, alveolar, nasal, intranasal, enteral, oral, intraoral, transoral, intestinal, rectal, intrarectal, transrectal, injection, infusion, intravenous, intraarterial, intramuscular, intracerebral, intraventricular, intracerebroventricular, intracardiac, subcutaneous, intraosseous, intradermal, intrathecal, intraperitoneal, intravesical, intracavernosal, intramedullar, intraocular, intracranial, transdermal, transmucosal, transnasal, inhalational, intracisternal, epidural, peridural, intravitreal, etc. The disclosed compositions can be used in and with any other therapy.

In another embodiment, one or more components of the solution can be provided as a “concentrate”, e.g., in a storage container (e.g., in a premeasured volume) ready for dilution, or in a soluble capsule ready for addition to a volume of water.

The foregoing formulations and administration methods are intended to be illustrative and not limiting. It will be appreciated that, using the teaching provided herein, other suitable formulations and modes of administration can be readily devised.

### 10. Combination Therapy

In one aspect of the disclosed methods, the pharmaceutical compositions can be administered alone or in combination with one or more additional therapeutic agents. The additional therapeutic agents are selected based on the disease or symptom to be treated. A description of the various classes of suitable pharmacological agents and drugs may be found in Goodman and Gilman, The Pharmacological Basis of Therapeutics, (11th Ed., McGraw-Hill Publishing Co.) (2005). For example, pharmaceutical compositions containing molecules can be administered in combination with one or more known therapeutic agents for treating wounds.

Examples of therapeutic agents that treat wounds injuries include, but are not limited to, anti-inflammatory agents, analgesic agents, anti-rheumatologic agents, laser devices, and immune modulating agents.

The molecules, compositions or pharmaceutical compositions can be administered in conjunction with or followed by any of the disclosed additional therapeutics.

The combination therapies can include administering the pharmaceutical composition and an additional therapeutic agent during the treatment cycle of a dosing regimen.

### D. Examples

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

Those skilled in the art will recognize, or be able to ascertain using no more than routine experimentation, many equivalents to the specific embodiments of the method and compositions described herein. Such equivalents are intended to be encompassed by the following claims.

### 1. Example 1

### Genetic Deletion of Cxcr2 Promotes Scarless Wound Healing

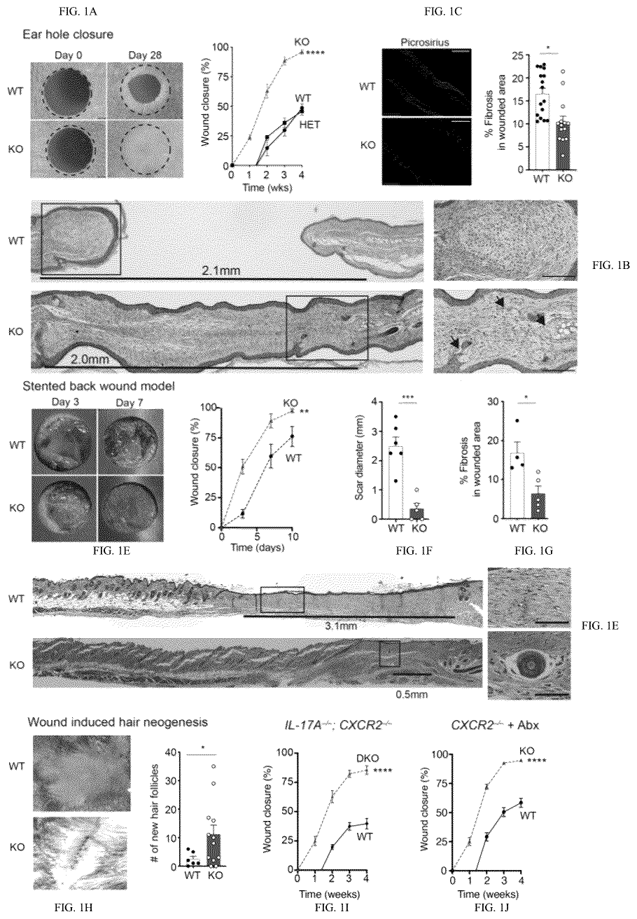

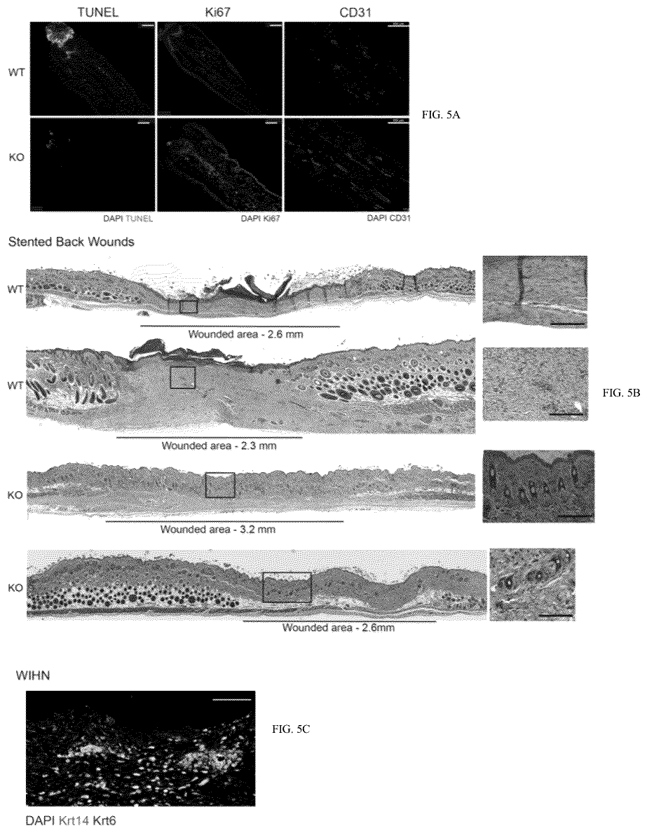

CXCR2-KO mice closed their ear holes faster and to a significantly smaller size compared to littermate WT or heterozygous controls (FIG. 1A). Complete ear hole closure occurred in 50% of CXCR2-KO mice and 0% in the littermate control mice. Notably, this is the highest frequency of complete ear hole closure in our experience using any regeneration-competent mouse strain (12, 15, 16). Hematoxylin and eosin (H&E) staining of wound edge tissue from WT control littermates revealed horizontally oriented fibroblasts and glassy thickened collagen, indicating tissue fibrosis and scar formation (FIG. 1B). Opposing cartilage end plates remained approximately 2 mm apart, confirming the absence of cartilage regeneration. By contrast, H&E staining on healed tissue from CXCR2-KO mice revealed normal tissue architecture, with return of hair follicles and sebaceous glands (FIG. 1B, black arrows denote regenerated structures). New islands of proliferating chondrocytes and a shortened distance between opposing cartilage end plates indicated cartilage regeneration. Fibrosis in the wounded area was measured with picrosirius red staining and significantly less fibrosis in CXCR2-KO healed tissue was found (FIG. 1C). Finally, Ki-67, CD31 and TUNEL staining on CXCR2-KO skin revealed increased proliferation, angiogenesis and decreased apoptosis, respectively compared to control littermates (FIG. 5A).

Stented dorsal back skin wounds on mice typically heal with scar formation (13). CXCR2-KO mice closed stented 6 mm dorsal back skin wounds faster than WT littermate controls (FIG. 1D). Histological analysis of wound edge tissue from WT control littermates revealed horizontally oriented fibroblasts, absence of secondary skin organs, and a scar diameter of ˜3 mm (FIGS. 1E and 5B). In contrast, injured CXCR2-KO skin demonstrated normal tissue architecture and return of hair follicles and sebaceous glands. Healed skin from CXCR2-KO mice developed 8-fold smaller scars compared to WT mice (FIG. 1F). CXCR2-KO mice also exhibited less fibrosis in their wounded areas compared to WT mice (FIG. 1G).

Finally, WIHN measures quality of wound healing through regeneration of hair follicles (14). Compared to control littermates, CXCR2-KO mice exhibited a 6-fold increase in the number of regenerated hair follicles (FIG. 1H). Immunohistochemistry with hair follicle specific marker genes (keratin-6+) confirmed the presence of new hair follicles (FIG. 5C). Taken together, three different skin injury models demonstrated that CXCR2-KO mice heal with faster wound closure, decreased scar formation, and improved hair regeneration.

### Improved Healing in CXCR2-KO Mice not Due to Increased IL-17A or Circulating Neutrophils

CXCR2-KO mice exhibit baseline increased circulating levels of IL-17A and neutrophils (7). Mice lacking both CXCR2 and IL-17A (CXCR2−/−; IL-17A−/−) were generated, and these mice continued to exhibit improved ear hole closure compared to littermate controls (FIG. 1I). Prior work showed that the gut microbiome regulated circulating neutrophil levels in CXCR2-KO mice, and antibiotic treatment would resolve this neutrophilia, (7). Antibiotic-treated CXCR2-KO mice healed as efficiently as control-treated CXCR2-KO mice (FIG. 1J). Thus, the improved healing phenotype in CXCR2-KO mice is not dependent on IL-17A, commensal bacteria, or increased circulating neutrophils.

### Cell-Specific Cxcr2 Knockout Mice Exhibit Partial Tissue Regeneration

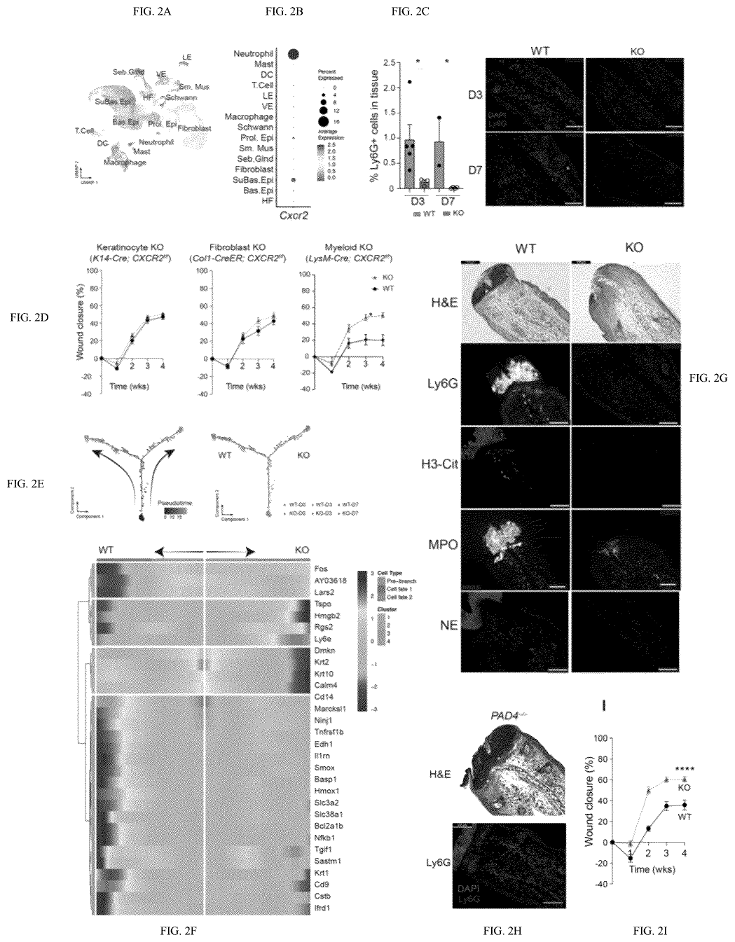

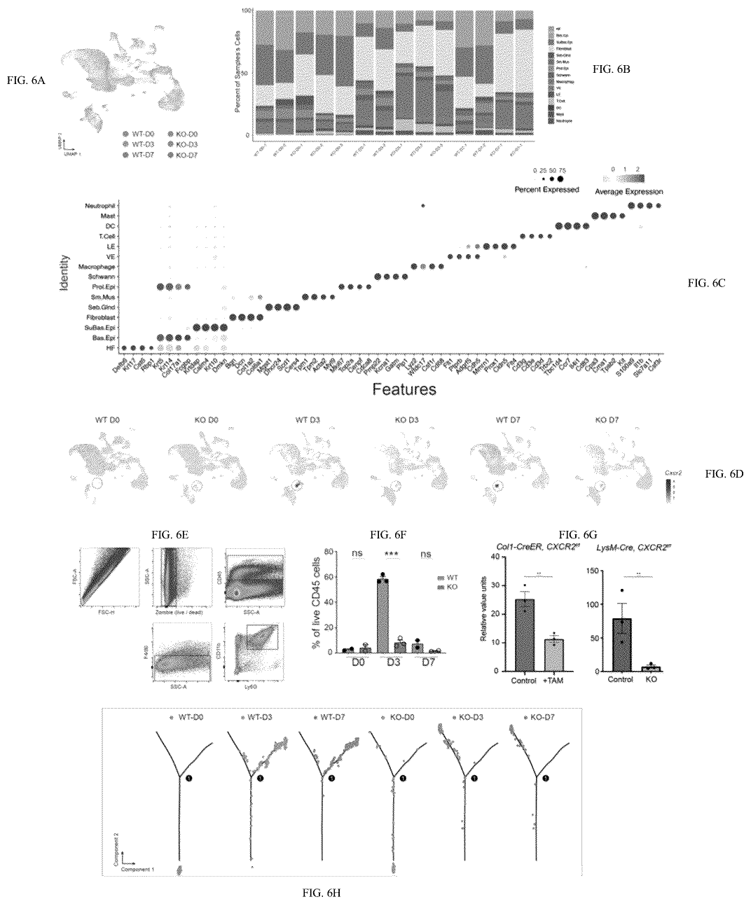

Single-cell RNA sequencing (scRNA-seq) was used on injured skin to identify cell types that express Cxcr2. Wound-edge tissue was collected from WT and CXCR2 KO mice at day 0, 3, and 7 after injury. 95,474 high-quality scRNA-seq profiles were generated (Table 1). Unsupervised clustering of scRNA-seq profiles identified 43 cell clusters, which were annotated to 15 cell types based on marker gene identification, lineage marker genes, and mapping to single-cell databases (FIGS. 2A and 6A-6C). Cxcr2 transcript was predominantly expressed in neutrophils and also found in keratinocytes, fibroblasts, and macrophages (FIGS. 2B and 6D). Immunostaining (Ly6G+) and flow cytometry on injured wound-edge skin confirmed increased recruitment of neutrophils in WT mice at day 3 and 7 after injury (FIGS. 2C and 6E-6F). Neutrophil recruitment was markedly reduced in CXCR2-KO mice.

TABEL 1
Data quality metrics of the single cell generated in the study

| | | | Median | Median | Median | Median |
|---|---|---|---|---|---|---|
| | Sequencing | Sequencing | Filtered genes | Filtered UMIs | value of | Filtered |
| Genotype | library ID | reads | per cell | per cell | mitoRatio | number of cells |
| Wild type | WT_D0_1 | 242,583,811 | 1,743 | 4,273 | 0.05 | 10496 |
| Wild type | WT_D0_2 | 349,793,518 | 1,390 | 3,094 | 0.05 | 17244 |
| Wild type | WT_D3_1 | 221,874,515 | 2,627 | 7,672 | 0.04 | 3957 |
| Wild type | WT_D3_2 | 306,483,461 | 3,059 | 9,907 | 0.04 | 4672 |
| Wild type | WT_D7_1 | 347,176,448 | 2,893 | 10,473 | 0.05 | 5345 |
| Wild type | WT_D7_2 | 244,227,745 | 3,003 | 12,211 | 0.04 | 6404 |
| Cxcx2 | KO_D0_1 | 385,756,642 | 662 | 1,245 | 0.04 | 16260 |
| Knockout | | | | | | |
| Cxcx2 | KO_D0_2 | 350,111,351 | 3,599 | 14,179 | 0.03 | 3527 |
| Knockout | | | | | | |
| Cxcx2 | KO_D0_3 | 372,145,807 | 3,289 | 13,019 | 0.03 | 6037 |
| Knockout | | | | | | |
| Cxcx2 | KO_D3_1 | 396,853,947 | 2,354 | 7,398 | 0.03 | 5117 |
| Knockout | | | | | | |
| Cxcx2 | KO_D3_2 | 307,234,127 | 2,730 | 8,488 | 0.03 | 3781 |
| Knockout | | | | | | |
| Cxcx2 | KO_D3_3 | 288,091,699 | 2,964 | 8,539 | 0.04 | 3348 |
| Knockout | | | | | | |
| Cxcx2 | KO_D7_1 | 308,703,388 | 3,291 | 9,961 | 0.05 | 4574 |
| Knockout | | | | | | |
| Cxcx2 | KO_D7_2 | 313,078,413 | 3,271 | 9,525 | 0.05 | 4712 |
| Knockout | | | | | | |

To identify which cell type(s) require loss of Cxcr2 to exhibit improved wound healing, mice lacking Cxcr2 specifically in keratinocytes (K14-Cre; CXCR2^{f/f}), fibroblasts (COL1A1-CreER; CXCR2^{f/f}), or myeloid cells (neutrophils and macrophages, LysM-Cre; CXCR2^{f/f}) were generated. Functional deletion by real-time PCR was confirmed (FIG. 6G). In the ear hole closure model, keratinocyte- or fibroblast-specific CXCR2-KO mice did not exhibit improved healing compared to control littermates (FIG. 2D). In contrast, myeloid-specific CXCR2-KO mice closed ear holes to a significantly smaller size (˜40%) compared to littermate controls (˜20%). However, none of the mice exhibited complete ear hole closure (FIG. 2D). Notably, ear hole closure speed was improved during weeks 1-2 before plateauing during weeks 3-4. Therefore, myeloid-specific Cxcr2 knockout mice partially recapitulated the improved healing phenotype of global CXCR2-KO.

### CXCR2+Neutrophils Secrete NETs to Promote Fibrosis and Scar Formation

Pseudotime analysis was used to assess gene expression changes within skin-resident WT and CXCR2-KO neutrophils. At day 3 and 7 after injury, neutrophils from WT and CXCR2-KO mice diverged along distinct paths (FIGS. 2E and 6H). Key differences in gene categories included, apoptosis (Ctsb, Tnfrsf1b), inflammation (Cd9, Cd14) and reactive oxygen species (Ifrdl, Hmoxl) (FIG. 2F). Neutrophil activation generally leads to secretion of neutrophil extracellular traps (NETs), and NET formation was assessed by immunostaining for citrullinated histone H3 (H3-Cit), myeloperoxidase (MPO), and neutrophil elastase (NE). Wound-edge tissue from WT mice collected on day 3 post injury exhibited exuberant NET formation, while wound-edge tissue from CXCR2-KO mice contained minimal NET formation (FIG. 2G). Wound scabs are thought to be composed of dried blood and cellular debris. In WT mice, neutrophils and NETs localized specifically to the wound scab and constituted a large portion of it (FIG. 2G). In contrast, CXCR2-KO mice did not develop any wound scabs.

To assess the role of NETs in wound healing, mice lacking PAD4−/−were obtained (henceforth known as PADI4-KO), which are specifically unable to generate NETs. These mice exhibited normal neutrophil homing (FIG. 2H). In the ear hole closure assay, they healed car holes to a smaller size (˜60%) compared to littermate controls (25%). None of the PADI4-KO mice exhibited complete ear hole closure (FIG. 21). Thus, PADI4-KO mice exhibited a partial regenerative phenotype similar to that of myeloid-specific CXCR2-KO mice. Thus, neutrophil secreted NETs promote fibrosis and scar formation during early time points after injury.

### Injured CXCR2-KO Skin Recruit More Anti-Inflammatory Macrophages at Early Time Points

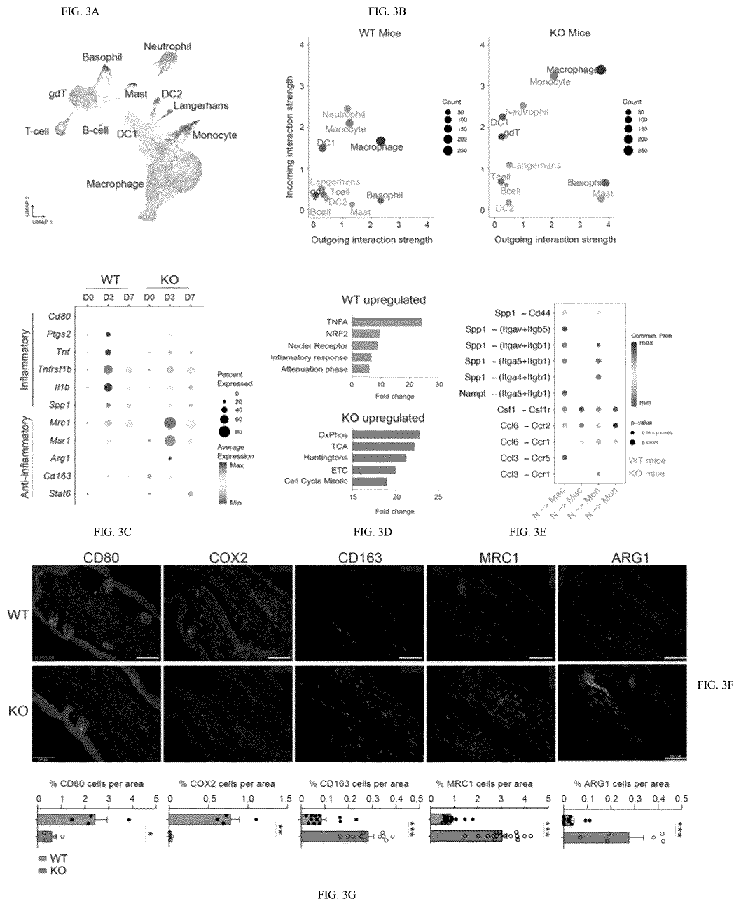

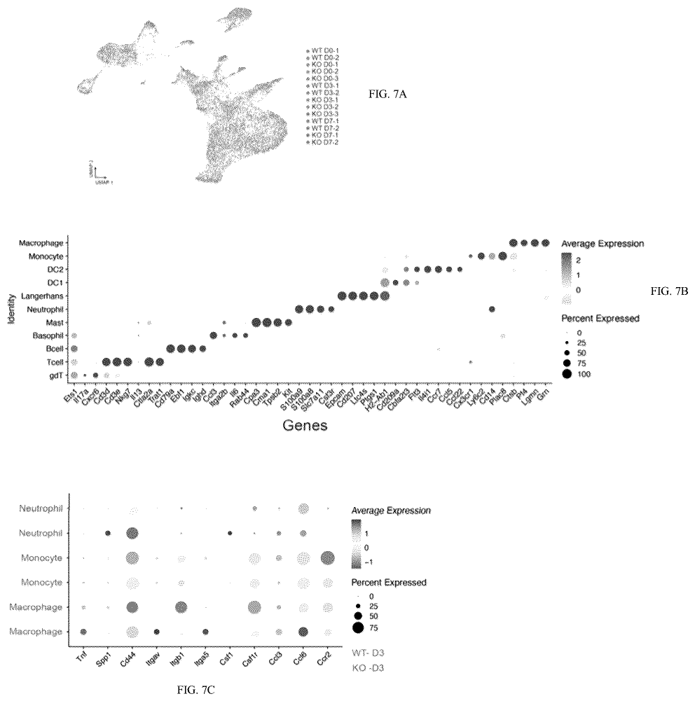

Since cell-specific CXCR2-KO mice did not completely phenocopy the global CXCR2-KO phenotype, immune cell populations were subclustered in our single-cell RNA sequencing dataset and identified eleven immune cell types in WT and CXCR2-KO mice (FIGS. 3A and 7A-7B). Global ligand-receptor analysis revealed signaling differences between WT and CXCR2-KO mice in macrophage and monocyte populations (FIG. 3B).

Over the past decade, functional heterogeneity and plasticity within macrophages has become established, and macrophages may adopt inflammatory and anti-inflammatory cell states. Injury induced WT macrophages to express inflammatory marker genes, including Cd80, Ptgs2, Tnf, Il-1b, and Spp1 (FIG. 3C). In contrast, injury induced CXCR2-KO macrophages to express anti-inflammatory macrophage marker genes, including Mrc1, Msr1, Arg1, Cd163, and Stat6 (FIG. 3C). Consistently, Gene Set Enrichment analysis (GSEA) in WT macrophages identified pathway changes consistent with an inflammatory phenotype, including TNF signaling, NRF2 activation, and inflammatory response (FIG. 3D). CXCR2-KO macrophages exhibited pathway changes consistent with an anti-inflammatory phenotype, including oxidative phosphorylation, electron transport chain (ETC) activation, and tricarboxylic acid cycle (TCA) activation (FIG. 3D). These changes may be dependent on different neutrophil interactions. Ligand-receptor analysis revealed that WT neutrophils secreted osteopontin (Spp1), Ccl3, and Ccr5, which were received by macrophages and monocytes. These interactions were absent in CXCR2-KO mice (FIGS. 3E and 7C).

Gene expression changes were validated by immunofluorescence on day 3 wound-edge tissue. WT skin exhibited increased expression of pro-inflammatory genes CD80 and COX2 (Ptgs2), whereas injured CXCR2-KO skin displayed increased expression of anti-inflammatory genes CD163, MRC1, and ARG1. Taken together, injury induced macrophages in WT and CXCR2-KO mice to adopt a pro-inflammatory and anti-inflammatory phenotype, respectively.

### Circulating Factor in CXCR2-KO Blood Promotes Scarless Tissue Regeneration

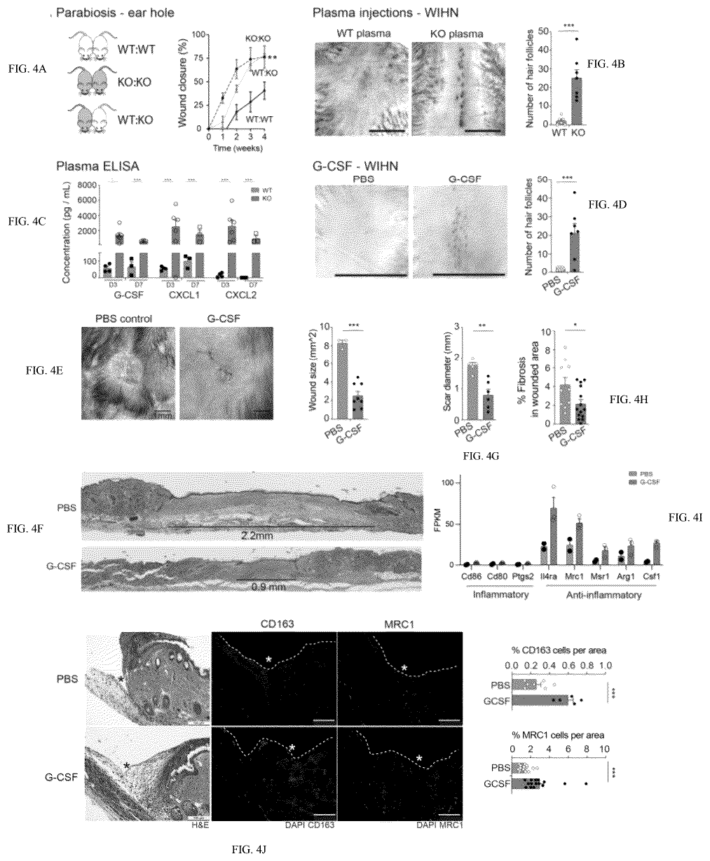

To assess whether a circulating factor in CXCR2-KO mice contributes to tissue regeneration, parabiosis pairs of WT: CXCR2-KO mice and control pairs (WT: WT and CXCR2-KO: CXCR2-KO) were generated. The ear hole injury model was performed to minimize further trauma to the back skin. As expected, CXCR2-KO: CXCR2-KO pairs closed ear holes faster and to a smaller size compared to WT: WT pairs (FIG. 4A). The WT: CXCR2-KO parabiosis pairs closed ear holes with a slower speed compared to the CXCR2-KO: CXCR2-KO control pairs but achieved the same final ear hole size (FIG. 4A, blue line vs. red line). Therefore, one or more circulating factor(s) in CXCR2-KO blood promotes scarless wound healing.

To assess whether the circulating factor(s) was a cell or protein, plasma from CXCR2-KO and WT (control) mice was collected and injected daily into the wound beds of WT mice undergoing WIHN for the first 3 days after injury (FIG. 4B). The WIHN model was used because the wound bed was more accessible. Strikingly, WT mice treated with CXCR2-KO plasma exhibited 5-fold increased hair follicle regeneration compared to control mice (FIG. 4B). Thus, a circulating protein in CXCR2-KO plasma improves tissue regeneration.

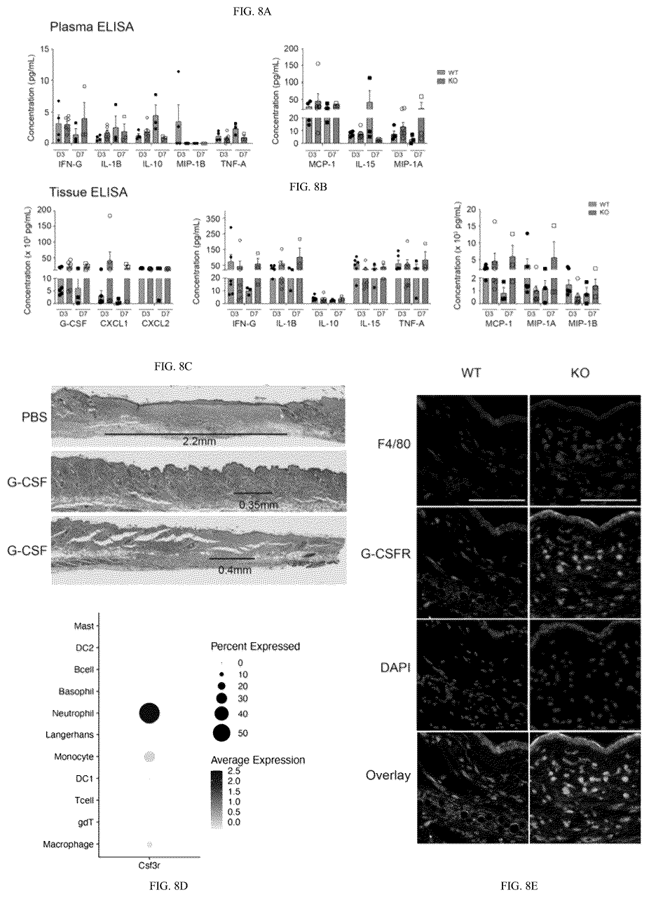

Luminex multianalyte cytokine analysis was performed on CXCR2-KO and littermate control plasma collected at day 3 and day 7 after injury. 12 major cytokines were assessed, including IFNg, IL-1b, TNFa, MCP-1, and IL-10. Consistent with prior studies, increased levels of CXCL1 and CXCL2, known ligands for CXCR2, were found in CXCR2-KO plasma (FIGS. 4C and 8A) (7). A 24-fold increase in G-CSF in injured CXCR2-KO plasma compared to injured WT plasma was found. Wound-edge tissue samples between WT and CXCR2-KO mice did not exhibit a similar induction of G-CSF, which suggests that increased circulating levels of G-CSF induction were not triggered by local injury (FIG. 8B).

### Exogeneous G-CSF Reduces Scar Formation and Improves Hair Follicle Regeneration

Next, recombinant G-CSF or PBS (control) was injected daily into the wound beds of WT mice undergoing WIHN for the first 3 days (FIG. 4D). G-CSF treated mice exhibited a 5-fold increase in hair follicle regeneration compared to control mice. This experiment was repeated in the stented back wound model. G-CSF-treated mice healed with >3-fold less scar formation compared to control mice (FIG. 4E). Histological analysis of wound edge tissue from PBS-treated WT mice revealed horizontally oriented fibroblasts, absence of secondary hair organs, and an average scar diameter of ˜1.8 mm (FIGS. 4F and 4G). In contrast, G-CSF-treated WT mice exhibited an average scar diameter of ˜0.5 mm. Healed skin from G-CSF-treated WT mice also contained less fibrosis than control skin (FIG. 4H). Taken together, exogenous G-CSF is sufficient to reduce scar formation and to promote complete tissue regeneration.

In our single-cell datasets, neutrophils and macrophages were the primary cell types in injured skin to express the G-CSF receptor (Csfr3) (FIG. 8D). CXCR2-KO mice exhibited reduced neutrophil recruitment to injured tissue. Immunofluorescence confirmed that macrophages in WT and CXCR2-KO injured skin expressed the G-CSF receptor (FIG. 8E). G-CSF directly polarized in vitro bone-marrow derived monocytes into an anti-inflammatory state (FIG. 4I) (17-19). Moreover, wounded tissue was collected at day 4 after injury in the stented back wound model from G-CSF-treated or PBS-treated WT mice. G-CSF-treated mice exhibited more anti-inflammatory macrophages (Cd163 and Mrc1) compared to PBS-treated mice (FIG. 4J). Taken together, G-CSF reduces scar formation and promotes tissue regeneration through polarization of infiltrating macrophages into an anti-inflammatory phenotype.

### DISCUSSION

In this work, CXCR2-KO mice exhibited robust scarless tissue regeneration across three different skin injury models. This regenerative ability is regulated by two distinct molecular mechanisms. 1) CXCR2 regulates neutrophil localization, and NETs secreted by CXCR2+neutrophils promote scar formation and fibrosis. 2) CXCR2-KO mice exhibit elevated levels of circulating G-CSF. G-CSF is sufficient to polarize macrophages into an anti-inflammatory phenotype and promote scarless tissue regeneration in WT mice.

Conventional wisdom says to leave wound scabs alone for optimal healing. Wound scabs were shown to be predominantly composed of neutrophils and NETs. Consistent with PADI4-KO mice exhibiting partial tissue regeneration, PADI4-KO mice were shown to modestly increase hair follicle regeneration 2.5-fold (20). These benefits are likely restricted to early time points, because neutrophils are only recruited during the first few days after injury. Actively removing wound scabs may reduce scar formation (21, 22).

How does the loss of CXCR2 result in increased circulating G-CSF? Neutrophil homeostasis is tightly regulated, and bone marrow stromal cells typically secrete G-CSF to allow neutrophils to exit the bone marrow (23). CXCR2 was shown to regulate neutrophil homeostasis by reducing circulating G-CSF levels through commensal bacteria-stimulated IL-17A (7). However, our improved healing phenotype in CXCR2-KO mice is not dependent on IL-17A or commensal bacteria (FIGS. 1H and 1I). CXCR2-KO mice also exhibit neutrophil hyperplasia within the bone marrow, and this may directly contribute to increased circulating levels of G-CSF.

Prior work demonstrated that complete depletion of macrophages prevents overall wound healing, but depletion at different time points results in different outcomes (24). Depletion of early-stage inflammatory macrophages reduced scar formation, and depletion of mid-stage macrophages prevented wound closure. Moreover, early- and late-stage wound macrophages are also tightly linked to specific metabolic profiles (25). Early-stage inflammatory wound macrophages are more glycolytic, and late-stage anti-inflammatory wound macrophages induce genes involved in TCA cycle and oxidative metabolism. CXCR2-KO wound beds were demonstrated to contain more macrophages that adopt an anti-inflammatory phenotype with activation of TCA cycle and oxidative metabolism genes during the early stage of wound healing. G-CSF also directly polarized macrophages into an anti-inflammatory phenotype to promote tissue regeneration. These results are consistent with prior work demonstrating that anti-inflammatory macrophages promote an anti-fibrotic response (26-30). Taken together, modulating macrophage activation states at early time points after injury represents a new strategy to promote scarless tissue regeneration.

Finally, a recent study demonstrated that exogenous complement factor H (CFH) reduces fibroblast-specific expression of Cxcl2, a CXCR2 ligand, to promote partial tissue regeneration (31). CFH led to reduced neutrophil recruitment and no changes in macrophage recruitment. This finding is consistent with our neutrophil-based mechanism of partial tissue regeneration.

More than 100 million new acute skin wounds are created annually, and ˜20 billion dollars are spent annually on treatment. Current treatment paradigms for acute wounds are lacking and represent a major unmet clinical need. Short courses of G-CSF are routinely and safely used in healthy volunteers who donate their stem cells for bone marrow transplantation (32). A pilot clinical trial tested systemic G-CSF in epidermolysis bullosa patients, a rare genetic skin disease with increased skin fragility, and found reduced blister counts and wound sizes (33).

### Materials and Methods

### Study Design

Predefined study components including rules for stopping data collection, data inclusion/exclusion criteria, and endpoint selection methods were used. Specific information is described in their relevant section. Presented data combines all experiments, and unless noted, all experiments were repeated at least 2 times independently. Animals used in this study were randomly assigned to experimental groups, and investigators were not blinded to allocation during experiments and outcome assessment unless noted in the text. Sample size justification for animal studies was based on preliminary experiments.

### Mice

Wild-type C57BL/6J (#000664), CXCR2−/−(#2724), CXCR2^{f/f} (#24638), K14-Cre (#4782), COL1A1-CreER (#27751), LysM-Cre (#4781), and IL-17+ (#35717) mice were used. All mice were group-housed in an animal facility on a 12-hour light/12-hour dark cycle with ad libitum access to water and normal chow.

### Injury Models

### Ear Hole Closure

For ear wounding, a standard 2 mm mechanical punch (Roboz, Gaithersburg, MD) was used to create a hole in the center of each outer ear (pinna). Ear hole diameter was measured using a dissection microscope (Nikon) in the horizontal and vertical directions on a weekly basis. Ears were excluded if there were signs of wound infection, tearing of the ear, or abnormal geometric shape. These criteria were pre-established. For the antibiotic experiments, the same protocol as previously published was used (11).

### Wound Induced Hair Neogenesis

1-cm² full-thickness skin wounds were made as previously described (10). 5-weeks later, de novo hair follicles were identified by whole-mount alkaline phosphatase staining of dermis preparations as previously described.

### Stented Small Back Wounds

6 mm disposable biopsy punch (Acuderm) was used to make two circular full thickness wounds on the dorsal back skin of mice. Silicon wound splints (Grace Bio-Labs) were sutured with 4-0 Nylon to prevent skin contracture. Wounds were dressed with a sterile occlusive dressing and monitored daily. Borders were monitored by frequently application of permanent marker.

### Parabiosis

Parabiosis surgery followed previously described procedures (39). Briefly, mirror-image incisions at the left and right flanks were made through the skin. Elbow and knee joints from each parabiont were sutured together with 3-0 Nylon, and the skin of each mouse was sutured with 4-0 Nylon to the skin of the adjacent parabiont. Each mouse was treated with subcutaneous normal saline, meloxicam (Putney), and buprenorphine hydrochloride (Butler Schein Animal Health) as directed for pain and monitored closely during recovery. 2-3 days after surgery, steristrips were placed over the sutures on the skin. For overall health, several recovery characteristics were analyzed at various times after surgery, including weight and grooming responses, and animals were excluded if they failed overall health inspection. The standard ear punch assay was administered one month after the parabiosis surgery.

### Histology and Immunohistochemistry

Standard histology and immunostaining protocols were performed, and investigators were blinded during histologic staining. 4% paraformaldehyde was used as the fixative. Briefly, immunohistochemical analysis was performed on 5-10 μm-thick sections of mouse skin. The following primary antibodies were used: rabbit monoclonal anti-Arginase-1 (93668, Cell Signaling Technology), rabbit polyclonal anti-CD80 (8679, ProSci), rabbit polyclonal anti-Histone H3 (ab5103, Abcam), goat polyclonal anti-MPO (AF3667, R&D Systems), Goat polyclonal anti-CD206 (AF2535, R&D Systems), rabbit monoclonal anti-F4/80 (70076, Cell Signaling Technology), rabbit polyclonal anti-COX2 (ab15191, Abcam), rabbit anti-iNOS (2982, Cell Signaling Technology), rabbit polyclonal anti-GCSFR (bs-2574R, Bioss) rabbit monoclonal anti-CD163 (68922, Cell Signaling Technology) and rat anti-Ly6G (551459, BD Pharmingen). The following secondary antibodies were used: goat polyclonal anti-Rabbit IgG (H+L) Alexa Fluor™ 488 (A-11008 Thermo-Fisher), goat polyclonal anti-Rat IgG (H+L) Alexa Fluor™ 647 (A-21247 Thermo-Fisher) and donkey polyclonal anti-rabbit IgG (H+L) Alexa Fluor™ 555 (A-31572 Thermo-Fisher). After staining, images were directly analyzed using a Leica Microsystems DM6 B microscope equipped with a DFC9000 Camera or Keyence imaging system. A minimum of 4-6 sections were stained per sample. Secondary only control was included for every experiment. Unwounded skin was included as a control for each antibody. Representative images were selected for figure panels. Immunofluorescent images were analyzed using FIJI (40). The signal for the protein of interest was calculated as the percent area occupied by the cells positive for the protein of interest within the tissue section.

### Picrosirius Red (PSR) Staining

The paraffin sections were first de-waxed and hydrated. Subsequently, the nuclei were stained with hematoxylin. Picrosirius red (Sigma-Aldrich, 365548) was then added for one hour. Following this, the slides were washed twice with acidified water (0.05% glacial acetic acid). Excess water was removed from the slides through vigorous shaking or by blotting with damp filter paper. To complete the process, the slides underwent dehydration in three changes of 100% ethanol, followed by clearing in xylene, and finally, mounting in a resinous medium. The PSR images were acquired on a Leica DM6B-Z microscope using a light polarizer equipped with a 32 mm quarter-wave plate and an ICT/P analyzer module. The acquired images were analyzed for fibrosis by quantifying the percent of collagen 1 (PSR) signal within the wounded tissue region. Representative images were selected for figure panels.

### Flow Cytometry

For immune cell recruitment experiments, freshly dissected tissue from the rim of healing wounds were manually dissociated with scissors and then incubated with Liberase TL (Roche) for 90 min at 37° C. After dissociation, cells were washed in PBS and resuspended in FACS buffer (PBS+0.05% NaN3+2% FBS). Neutrophils were quantified using a modified version of a previously described flow cytometry gating strategy (41). Cells were stained with Zombie Green™ cell viability dye (423111, Zombie Green Biolegend) for 15 minutes in the dark at room temperature. Cells were pretreated with Fc-blocking agent TruStain FcX anti-mouse CD16/32 (101320, clone 93) and subsequently stained with the following monoclonal antibodies: CD45 (103130, clone 30-F11, BioLegend), F4/80 (123149, clone BM8, BioLegend), CD11b (101218, clone M1/70, BioLegend), GR-1 (127617, clone 1A8, BioLegend). Samples were acquired on a four-laser BD LSRII flow cytometer and all sample data was analyzed using FloJo software version 10.8.1 (BD).

### Luminex ELISA Assay

The quantification of chemokine/cytokines from mouse plasma and tissue protein extracts was performed using a Luminex™ 100 system at the University of Maryland SOM Cytokine Core Laboratory (CCL). For each assay, at least two technical replicates were used, and the number of biological replicates is mentioned in the individual data figure legends. The following chemokine/cytokines used were in the assay: IL-1beta, IL-10, TNF-alpha, G-CSF, IFN-gamma, IL-15, MCP-1, MIP-1 alpha, MIP-2, KC and MIP-1 beta.

### Bulk RNA Library Preparation

Monocytes were lysed immediately by adding TRIzol LS reagent. The samples were vortexed for 20 seconds, 0.2×volumes of chloroform was added, tubes were mixed by inverting and samples were centrifuged at 13,000 rpm at 4° C. for 15 min. The aqueous phase was then purified and the RNA-seq library were made using NEBNext® Low Input RNA Library Prep Kit (E6420S/L), following the manufacturer's instructions.

### Real-Time RT-PCR

Freshly dissected tissue was manually dissociated with scissors, collected in TRI Regcant (Zymo) and then mechanically disrupted (Fastprep 24, Lysing Matrix D, MP Bio). Total RNA was isolated by Direct-Zol RNA MicroPrep (Zymo). RNA concentration was measured by Nanodrop 1000 (Thermo Scientific). cDNA synthesis was performed with Maxima Reverse Transcriptase (Thermo Scientific) or Superscript IV VILO (Thermo Scientific) per manufacturer's instructions. One-step quantitative RT-PCR was performed and analyzed using an ABI ViiA7 Real-Time PCR detection system (Applied Biosystems) with TaqMan one-step RT-PCR Master Mix Reagents.

### Single-Cell Gene RNA-Seq Library Preparation and Sequencing

Freshly dissected tissue from the rim of healing wounds was manually dissociated with scissors in a serum free RPMI 1640 media with DNase I (0.2 mg/mL, 12633012, Thermo Fisher Scientific), 20 mM HEPES and 0.25 mg/ml Liberase TL (5401020001, Roche). The suspension was incubated for 90 minutes at 37° C. The digestion was stopped by adding 100 μl FBS and 3 μl of 0.5 M EDTA and filtered through a 70-mm cell strainer (22-363-548, Fisher Scientific). The cells were pelleted and washed twice with PBS containing 1% BSA. Finally, cells were resuspended in PBS containing 0.04% BSA and an aliquot was taken for counting. The scRNA-seq was performed using 10λ Chromium 3 v3.1 kit (1000268, 10× Genomics). The sequencing libraries were prepared per manufacturer's protocol and sequenced 2×100 bp paired-end run on the Illumina HiSeq2000/HiSeq2500 platforms at the BGI America. The raw and processed sequencing data details are given in Table 1.

### Computational Analysis

scRNA-Seq Data Analysis

The scRNA sequencing data was mapped to the GRCm38 reference genome to generate gene count and cell barcode matrices using the “cellranger count” function from the cellranger pipeline (version 5.0.1, 10× Genomics). All downstream analysis steps were performed using the R package Seurat (44) (ver. 4.3.0, https://github.com/satijalab/seurat) unless otherwise noted. In brief, seurat functions ‘Read10X’ and ‘CreateSeuratObject’ were used to import and create a merged Seurat object from all filtered feature barcode matrices generated by the cellranger pipeline. Cells with less <250 genes, <500 UMI, <0.80 log 10 Genes per UMI, and more than 10% mitochondrial reads were excluded from the merged Seurat object for further analysis. Genes that were detected in less than 10 cells were also discarded. DoubletFinder was used to identify potential cell doublets as a final quality control (45). To determine and regress out the effect of cell cycle, each cell was given a cell cycle phase score using the Seurat function ‘CellCycleScoring’ (46). The data was then log-normalized and scaled by linear regression against the number of reads. The Find VariableFeatures function followed by SelectIntegrationFeatures function (nfeatures=3000) were used to identify variable genes from merged Seurat object. For cross-tissue data integration and batch correction, ‘FindIntegrationAnchors’ and ‘IntegrateData’ were applied to the merged Seurat object. Dimensionality reduction was performed using the RunPCA and RunUMAP function generated UMAP plots. Next, Louvain clustering was performed with the ‘FindClusters’ function using the first 40 PCs and at resolution 1.4. The ElbowPlot function in Seurat, visual inspection of DimHeatmap plots at different dimensions and R package clustree were used to choose an optimum number of dimensions and resolution.

### Cell Type Annotation

Two complementary approaches were used to annotate the identities of different cell clusters: (1) the expression of lineage-specific marker genes identified from previously published single-cell RNA-Seq studies was checked in our query cluster marker genes list and in differentially expressed genes of the query cluster. (2) An unbiased cell type recognition method named SingleR (R package) was applied (47), which leverages mapping of the genes from the query cluster to the reference transcriptomic datasets of known cell types such as BlueprintEncode (48), MonoccoImmune reference (49), and Database of Immune Cell Expression (DICE) data (50). SingleR was first applied to determine if the predicted annotations were consistent with our findings and then assigned the identity to the cluster. The sample statistics and marker gene dot plots were made by using dittoSeq (v 1.4.1). The uniform manifold approximation and projection (UMAP) was applied to visualize the single cell transcriptional profile in 2D space based on the SNN graph described above (51). Other bar plots, boxplots, violin plots and heatmaps were generated by customized R code through ggplot2 (v3.2.1, R package) (52).

### Functional Enrichment Analysis

SCPA was used to assess gene set enrichment in both wild-type and knockout conditions, leveraging the Molecular Signatures Database (MSigDB), KEGG pathway, and Reactome pathway database (55). From this analysis, pathways with a Qval>4, where Qval signifies statistical significance (defined as sqrt (-log 10 (Bonferroni-adjusted p-value))), were chosen. Subsequently, the pathways were visualized with the most significant fold change (FC) enrichment scores.

### Pseudotime

The single cell pseudotime trajectory analysis was performed using the Monocle2 R package (v 2.18.0) 52. The WT and KO neutrophils Seurat object were used for the analysis. The integrated object was used as an input for pseudotime analysis, and genes expressed in at least 5% of the cells were selected to construct the pseudotime trajectory. Following dimensionality reduction using PCA and tSNE method, the densityPeak algorithm was run to cluster cells based on each cell's local density (P) and the nearest distance (Δ). Default values were chosen for parameters of the DDRTree method and visualization of dynamically expressed genes along the pseudotime was performed using the ‘plot genes_in_pseudotime’ function with the default parameters.

### Bulk RNA-Seq Analysis

Fastq files were aligned to the mm10 reference genome using STAR_2.4.0 in basic two Pass mode using the “Encode” options as specified in the manual. Reads overlapping with annotated genes (Ensembl build mm10) were counted using the summarizeOverlaps function from the R package “GenomicAlignments” in strand-specific, paired-end mode. FPKM (Fragments per kilobase per million mapped fragments) counts was estimated using DESeq2.

### Statistics

For in vivo time courses comparing hole size, data were each analyzed using 2-way analysis of variance (ANOVA) using a temporal main effect, a main effect comparing treatment, and an interaction of the two main effects. For tests such that the two-way ANOVA indicates significant time-treatment interactions, additional 2-tailed Student's t test was used, with P values of less than 0.05 considered significant. The conclusions for the test between treatments using the two methods are identical. 2-tailed Student's t test was used to determine significance, with P values of less than 0.05 considered significant. Higher levels of significance are indicated by the following: **P<0.01, ***P<0.001, and ****P<0.0001 in the text.

### 2. Example 2

A cutaneous neuroimmune signaling pathway regulated by activation of TRPA1 receptor on skin sensory nerves promotes regenerative wound healing (Wei et al., 2020, Science Immunology).

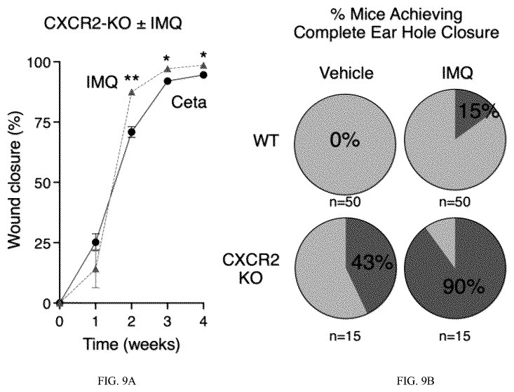

To assess whether the CXCR2-mediated pathway utilized the same molecular mechanism, TRPA1-agonist imiquimod 5% cream was rubbed on skin 5 days per week, beginning one week prior to ear hole injury in WT and CXCR2-KO mice. Imiquimod-treated mice closed their ear holes even more efficiently than control-treated mice (FIG. 9A). Imiquimod-treated CXCR2-KO mice achieved complete ear hole closure at a 90% frequency, which was >2-fold higher than CXCR2-KO mice (FIG. 9B, bottom graphs). Indeed, this 90% complete closure rate in imiquimod-treated CXCR2-KO mice is greater than the individual imiquimod or CXCR2 components combined.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

### REFERENCES

1. S. A. Eming, P. Martin, M. Tomic-Canic, Wound repair and regeneration: mechanisms, signaling, and translation. Sci Transl Med 6, 265sr266 (2014).
2. L. Guenin-Mace, P. Konieczny, S. Naik, Immune-Epithelial Cross Talk in Regeneration and Repair. Annu Rev Immunol 41, 207-228 (2023).
3. G. C. Gurtner, S. Werner, Y. Barrandon, M. T. Longaker, Wound repair and regeneration. Nature 453, 314-321 (2008).
4. P. Murawala, E. M. Tanaka, J. D. Curric, Regeneration: the ultimate example of wound healing. Semin Cell Dev Biol 23, 954-962 (2012).
5. A. W. Seifert, J. R. Monaghan, S. R. Voss, M. Maden, Skin regeneration in adult axolotls: a blueprint for scar-free healing in vertebrates. PLOS One 7, c32875 (2012).
6. J. W. Godwin, A. R. Pinto, N. A. Rosenthal, Macrophages are required for adult salamander limb regeneration. Proc Natl Acad Sci USA 110, 9415-9420 (2013).
7. J. Mei et al., Cxcr2 and Cxc15 regulate the IL-17/G-CSF axis and neutrophil homeostasis in micc. J Clin Invest 122, 974-986 (2012).
8. K. Tateda et al., Chemokine-dependent neutrophil recruitment in a murine model of Legionella pneumonia: potential role of neutrophils as immunoregulatory cells. Infect Immun 69, 2017-2024 (2001).
9. W. C. Tsai et al., CXC chemokine receptor CXCR2 is essential for protective innate host response in murine Pseudomonas acruginosa pneumonia. Infect Immun 68, 4289-4296 (2000).
10. R. M. Devalaraja et al., Delayed wound healing in CXCR2 knockout mice. J Invest Dermatol 115, 234-244 (2000).
11. R. D. Galiano, J. t. Michaels, M. Dobryansky, J. P. Levine, G. C. Gurtner, Quantitative and reproducible murine model of excisional wound healing. Wound Repair Regen 12, 485-492 (2004).
12. K. Bedelbacva et al., Lack of p21 expression links cell cycle control and appendage regeneration in mice. Proc Natl Acad Sci USA 107, 5845-5850 (2010).
13. S. Mascharak et al., Multi-omic analysis reveals divergent molecular events in scarring and regenerative wound healing. Cell Stem Cell 29, 315-327 e316 (2022).
14. M. Ito et al., Wnt-dependent de novo hair follicle regeneration in adult mouse skin after wounding. Nature 447, 316-320 (2007).
15. T. H. Leung, E. R. Snyder, Y. Liu, J. Wang, S. K. Kim, A cellular, molecular, and pharmacological basis for appendage regeneration in mice. Genes Dev 29, 2097-2107 (2015).
16. M. A. Nishiguchi, C. A. Spencer, D. H. Leung, T. H. Leung, Aging Suppresses Skin-Derived Circulating SDFI to Promote Full-Thickness Tissue Regeneration. Cell Rep 24, 3383-3392 c3385 (2018).
17. M. Hollmen et al., G-CSF regulates macrophage phenotype and associates with poor overall survival in human triple-negative breast cancer. Oncoimmunology 5, c1115177 (2016).
18. I. Karagiannidis et al., G-CSF and G-CSFR Induce a Pro-Tumorigenic Macrophage Phenotype to Promote Colon and Pancreas Tumor Growth. Cancers (Basel) 12, (2020).
19. Q. Wen et al., G-CSF-induced macrophage polarization and mobilization may prevent acute graft-versus-host disease after allogeneic hematopoietic stem cell transplantation. Bone Marrow Transplant 54, 1419-1433 (2019).
20. E. Wier et al., Neutrophil extracellular traps impair regeneration. J Cell Mol Med 25, 10008-10019 (2021).
21. W. Lec et al., Neutrophils facilitate ovarian cancer premetastatic niche formation in the omentum. J Exp Med 216, 176-194 (2019).
22. C. W. Steele et al., CXCR2 Inhibition Profoundly Suppresses Metastases and Augments Immunotherapy in Pancreatic Ductal Adenocarcinoma. Cancer Cell 29, 832-845 (2016).
23. R. B. Day, D. C. Link, Regulation of neutrophil trafficking from the bone marrow. Cell Mol Life Sci 69, 1415-1423 (2012).
24. T. Lucas et al., Differential roles of macrophages in diverse phases of skin repair. J Immunol 184, 3964-3977 (2010).
25. S. Willenborg, L. Injarabian, S. A. Eming, Role of Macrophages in Wound Healing. Cold Spring Harb Perspect Biol 14, (2022).
26. A. Kasuya, T. Ito, Y. Tokura, M2 macrophages promote wound-induced hair neogenesis. J Dermatol Sci 91, 250-255 (2018).
27. Q. Cao, D. C. Harris, Y. Wang, Macrophages in kidney injury, inflammation, and fibrosis. Physiology (Bethesda) 30, 183-194 (2015).
28. T. A. Wynn, K. M. Vannella, Macrophages in Tissue Repair, Regeneration, and Fibrosis. Immunity 44, 450-462 (2016).
29. K. Klinkert et al., Selective M2 Macrophage Depletion Leads to Prolonged Inflammation in Surgical Wounds. Eur Surg Res 58, 109-120 (2017).
30. M. G. Rohani et al., MMP-10 Regulates Collagenolytic Activity of Alternatively Activated Resident Macrophages. J Invest Dermatol 135, 2377-2384 (2015).
31. K. L. Mack et al., Allele-specific expression reveals genetic drivers of tissue regeneration in mice. Cell Stem Cell 30, 1368-1381 c1366 (2023).
32. S. A. Grupp et al., Use of G-CSF in matched sibling donor pediatric allogeneic transplantation: a consensus statement from the Children's Oncology Group (COG) Transplant Discipline Committee and Pediatric Blood and Marrow Transplant Consortium (PBMTC) Executive Committee. Pediatr Blood Cancer 46, 414-421 (2006).
33. J. D. Finc, B. Manes, H. Frangoul, Systemic granulocyte colony-stimulating factor (G-CSF) enhances wound healing in dystrophic epidermolysis bullosa (DEB): Results of a pilot trial. J Am Acad Dermatol 73, 56-61 (2015).
34. Shen, G. Y., Park, I. H., Song, Y. S., Joo, H. W., Lec, Y., Shin, J. H., Kim, K. S., and Kim, H. (2016). Local injection of granulocyte-colony stimulating factor accelerates wound healing in a rat excisional wound model. Tissue Eng Regen Med 13, 297-303.
35. Day, R. B., and Link, D. C. (2012). Regulation of neutrophil trafficking from the bone marrow. Cell Mol Life Sci 69, 1415-1423.
36. Mack, K. L., Talbott, H. E., Griffin, M. F., Parker, J. B. L., Guardino, N. J., Spielman, A. F., Davitt, M. F., Mascharak, S., Downer, M., Morgan, A., et al. (2023). Allele-specific expression reveals genetic drivers of tissue regeneration in mice. Cell Stem Cell 30, 1368-1381 c1366.
37. Grupp, S. A., Frangoul, H., Wall, D., Pulsipher, M. A., Levine, J. E., and Schultz, K. R. (2006). Use of G-CSF in matched sibling donor pediatric allogeneic transplantation: a consensus statement from the Children's Oncology Group (COG) Transplant Discipline Committee and Pediatric Blood and Marrow Transplant Consortium (PBMTC) Executive Committee. Pediatr Blood Cancer 46, 414-421.
38. Fine, J. D., Manes, B., and Frangoul, H. (2015). Systemic granulocyte colony-stimulating factor (G-CSF) enhances wound healing in dystrophic epidermolysis bullosa (DEB): Results of a pilot trial. J Am Acad Dermatol 73, 56-61.
39. Spencer, C. A., and Leung, T. H. (2019). Research Techniques Made Simple: Parabiosis to Elucidate Humoral Factors in Skin Biology. J Invest Dermatol 139, 1208-1213 e1201. 10.1016/j.jid.2019.03.1134.
40. Schindelin, J., Arganda-Carreras, I., Frise, E., Kaynig, V., Longair, M., Pietzsch, T., Preibisch, S., Rueden, C., Saalfeld, S., Schmid, B., et al. (2012). Fiji: an open-source platform for biological-image analysis. Nat Methods 9, 676-682.
41. Ubags, N. D. J., and Suratt, B. T. (2018). Isolation and Characterization of Mouse Neutrophils. Methods Mol Biol 1809, 45-57.
42. Stuart, T., Butler, A., Hoffman, P., Hafemeister, C., Papalexi, E., Mauck, W. M., 3rd, Hao, Y., Stocckius, M., Smibert, P., and Satija, R. (2019). Comprehensive Integration of Single-Cell Data. Cell 177, 1888-1902 e1821.
43. McGinnis, C. S., Murrow, L. M., and Gartner, Z. J. (2019). DoubletFinder: Doublet Detection in Single-Cell RNA Sequencing Data Using Artificial Nearest Neighbors. Cell Syst 8, 329-337 e324.
44. Tirosh, I., Venteicher, A. S., Hebert, C., Escalante, L. E., Patel, A. P., Yizhak, K., Fisher, J. M., Rodman, C., Mount, C., Filbin, M. G., et al. (2016). Single-cell RNA-seq supports a developmental hierarchy in human oligodendroglioma. Nature 539, 309-313.
45. Aran, D., Looncy, A. P., Liu, L., Wu, E., Fong, V., Hsu, A., Chak, S., Naikawadi, R. P., Wolters, P. J., Abate, A. R., et al. (2019). Reference-based analysis of lung single-cell
46. sequencing reveals a transitional profibrotic macrophage. Nat Immunol 20, 163-172. Becht, E., McInnes, L., Healy, J., Dutertre, C. A., Kwok, I. W. H., Ng, L. G., Ginhoux, F., and Newell, E. W. (2018). Dimensionality reduction for visualizing single-cell data using UMAP. Nat Biotechnol. 10.1038/nbt.4314.
47. Wickham, H. (2016). ggplot2: Elegant Graphics for Data Analysis. Use R!, 2nd ed. Springer International Publishing: Imprint: Springer.
48. Bibby, J. A., Agarwal, D., Freiwald, T., Kunz, N., Merle, N. S., West, E. E., Singh, P., Larochelle, A., Chinian, F., Mukherjee, S., et al. (2022). Systematic single-cell pathway analysis to characterize early T cell activation. Cell Rep 41, 111697.

1. A pharmaceutical composition comprising Granulocyte Colony-Stimulating Factor (G-CSF), a G-CSF receptor agonist or a combination thereof and a pharmaceutically acceptable carrier, excipient or diluent.

2. The pharmaceutical composition of claim 1, wherein the pharmaceutical composition is formulated in a solid, semi-solid or liquid dosage form.

3. The pharmaceutical composition of claim 2, wherein the pharmaceutical composition is formulated in a topical ointment, cream, lotion or spray.

4. The pharmaceutical composition of claim 1, wherein the composition comprises G-CSF.

5. The pharmaceutical composition of claim 1, wherein the G-CSF receptor agonist is myelopoietin, LG7455, a GCSF receptor agonist antibody or a combination thereof.

6.-10. (canceled)

11. A method for treating wound healing pathologies or for promoting wound healing in a subject having a wound healing pathology, the method comprising the step of administering to the subject a composition comprising a therapeutically effective amount of Granulocyte Colony-Stimulating Factor (G-CSF), a G-CSF receptor agonist or a combination thereof.

12. The method of claim 11, wherein said step of administering is performed during spreading stage of scar formation.

13. The method of claim 11, wherein the Granulocyte Colony-Stimulating Factor (G-CSF), a G-CSF receptor agonist or a combination thereof is administered by topical administration.

14. (canceled)

15. The method of claim 11, wherein the Granulocyte Colony-Stimulating Factor (G-CSF), a G-CSF receptor agonist or a combination thereof is administered by intradermal or subepidermal administration or intralesional injection.

16. (canceled)

17. The method of claim 11, wherein the wound is an abrasion, an avulsion, a burn, a laceration or a surgical wound.

18. The method of claim 11, wherein the composition comprises G-CSF.

19. The method of claim 11, wherein the G-CSF receptor agonist is myelopoietin, LG7455, a GCSF receptor agonist antibody or a combination thereof.

20.-24. (canceled)

25. The method of claim 11, wherein the composition further comprises rosemary extract or carnosic acid.

26. A method of regenerating tissue in a subject in need thereof, the method comprising the step of administering to the subject a composition comprising a therapeutically effective amount of Granulocyte Colony-Stimulating Factor (G-CSF), a G-CSF receptor agonist or a combination thereof.

27. The method of claim 26, wherein the Granulocyte Colony-Stimulating Factor (G-CSF), a G-CSF receptor agonist or a combination thereof is administered by topical administration.

28. (canceled)

29. The method of claim 26, wherein the Granulocyte Colony-Stimulating Factor (G-CSF), a G-CSF receptor agonist or a combination thereof is administered by intradermal or subepidermal administration or intralesional injection.

30. (canceled)

31. The method of claim 26, wherein the composition comprises G-CSF.

32. The method of claim 26, wherein the G-CSF receptor agonist is myelopoietin, LG7455, a GCSF receptor agonist antibody or a combination thereof.

33.-37. (canceled)

38. The method of claim 26, wherein the composition further comprises rosemary extract or carnosic acid.

39.-92. (canceled)
